(19) United States
(12) Patent Application Publication
(10) Pub. No.: US 2012/0295961 A1
(43) Pub. Date: Nov. 22, 2012

(54) MODULATION OF HEPATITIS B VIRUS (HBV) EXPRESSION

(76) Inventors: ERIC E. SWAYZE, ENCINITAS, CA (US); ROBERT K. HAMATAKE, DURHAM, NC (US)

(21) Appl. No.: 13/452,451

(22) Filed: Apr. 20, 2012

Related U.S. Application Data
(60) Provisional application No. 61/478,041, filed on Apr. 21, 2011.

(51) Int. Cl. A61K 31/7088; A61P 35/00; A61P 29/00; A61P 41/00; C07H 21/04; A61P 1/16

(52) U.S. Cl. 514/44 R; 536/23.1

(57) ABSTRACT

Disclosed herein are antisense compounds and methods for decreasing HBV mRNA, DNA and protein expression. Such methods, compounds, and compositions are useful to treat, prevent, or ameliorate HBV-related diseases, disorders or conditions.

### RELATED APPLICATIONS

This application claims priority under 35 USC 119(e) to U.S. Provisional Patent Application No. 61/478,041 filed Apr. 21, 2011, which is incorporated herein by reference in its entirety.

### SEQUENCE LISTING

The present application is being filed along with a Sequence Listing in electronic format. The Sequence Listing is provided as a file entitled BIOL0152USSEQ.txt created Apr. 18, 2012, which is approximately 12 Kb in size. The information in the electronic format of the sequence listing is incorporated herein by reference in its entirety.

### FIELD

In certain embodiments provided are methods, compounds, and compositions for inhibiting expression of hepatitis B virus (HBV) mRNA and protein in an animal. Such methods, compounds, and compositions are useful to treat, prevent, or ameliorate HBV-related diseases and disorders.

### BACKGROUND

Hepatitis B is a viral disease transmitted parenterally by contaminated material such as blood and blood products, contaminated needles, sexually and vertically from infected or carrier mothers to their offspring. It is estimated by the World Health Organization that more than 2 billion people have been infected worldwide, with about 4 million acute cases per year, 1 million deaths per year, and 350-400 million chronic carriers (World Health Organization: Geographic Prevalence of Hepatitis B Prevalence, 2004. http://www.who.int/vaccines-surveillance/graphics/htmls/hepbprev.htm).

The virus, HBV, is a double-stranded hepatotropic virus which infects only humans and non-human primates. Viral replication takes place predominantly in the liver and, to a lesser extent, in the kidneys, pancreas, bone marrow and spleen (Hepatitis B virus biology. Microbiol Mol Biol Rev. 64: 2000; 51-68.). Viral and immune markers are detectable in blood and characteristic antigen-antibody patterns evolve over time. The first detectable viral marker is HBsAg, followed by hepatitis B e antigen (HBeAg) and HBV DNA. Titers may be high during the incubation period, but HBV DNA and HBeAg levels begin to fall at the onset of illness and may be undetectable at the time of peak clinical illness (Hepatitis B virus infection—natural history and clinical consequences. N Engl J Med. 350: 2004; 1118-1129). HBeAg is a viral marker detectable in blood and correlates with active viral replication, and therefore high viral load and infectivity (Hepatitis B e antigen—the dangerous end game of hepatitis B. N Engl J Med. 347: 2002; 208-210). The presence of anti-HBsAb and anti-HBcAb (IgG) indicates recovery and immunity in a previously infected individual

Currently the recommended therapies for chronic HBV infection by the American Association for the Study of Liver Diseases (AASLD) and the European Association for the Study of the Liver (EASL) include interferon alpha (INFα), pegylated interferon alpha-2a (Peg-IFN2a), entecavir, and tenofovir. The nucleoside and nucleotide therapies, entecavir and tenofovir, are successful at reducing viral load, but the rates of HBeAg seroconversion and HBsAg loss are even lower than those obtained using IFNα therapy. Other similar therapies, including lamivudine (3TC), telbivudine (LdT), and adefovir are also used, but for nucleoside/nucleotide therapies in general, the emergence of resistance limits therapeutic efficacy.

Thus, there is a need in the art to discover and develop new anti-viral therapies. Additionally, there is a need for new anti-HBV therapies capable of increasing HBeAg and HBsAg seroconversion rates. Recent clinical research has found a correlation between seroconversion and reductions in HBeAg (Fried et al (2008) Hepatology 47:428) and reductions in HBsAg (Moucari et al (2009) Hepatology 49:1151). Reductions in antigen levels may have allowed immunological control of HBV infection because high levels of antigens are thought to induce immunological tolerance. Current nucleoside therapies for HBV are capable of dramatic reductions in serum levels of HBV but have little impact on HBeAg and HBsAg levels.

Antisense technology is emerging as an effective means for reducing the expression of specific gene products and may therefore prove to be uniquely useful in a number of therapeutic, diagnostic, and research applications for the modulation of HBV expression (See U.S. Patent Publication Nos. 2008/0039418 and 2007/0299027). Antisense therapy differs from nucleoside therapy in that it can directly target the transcripts for the HBV antigens and thereby reduce serum HBeAg and HBsAg levels. Because of the multiple, overlapping transcripts produced upon HBV infection, there is also an opportunity for a single antisense oligomer to reduce HBV DNA in addition to both HBeAg and HBsAg. Therefore, antisense technology is emerging as an effective means for reducing the expression of certain gene products and may therefore prove to be uniquely useful in a number of therapeutic, diagnostic, and research applications for the modulation of HBV.

### SUMMARY

Provided herein are methods, compounds, and compositions for modulating expression of HBV mRNA and protein. In certain embodiments, compounds useful for modulating expression of HBV mRNA and protein are antisense compounds. In certain embodiments, the antisense compounds are antisense oligonucleotides.

In certain embodiments, modulation can occur in a cell or tissue. In certain embodiments, the cell or tissue is in an animal. In certain embodiments, the animal is a human. In certain embodiments, HBV mRNA expression is reduced. In certain embodiments, HBV DNA levels are reduced. In certain embodiments, HBV protein levels are reduced. In certain embodiments, HBV antigen levels are reduced. In certain embodiments, HBV s-antigen (HBsAg) levels are reduced. In certain embodiments, HBV e-antigen (HBeAg) levels are reduced. Such reduction can occur in a time-dependent manner or in a dose-dependent manner.

In certain embodiments, compounds for modulating expression of HBV mRNA and protein are chimeric gapmers provided herein. In certain embodiments, the compounds are 16mer gapmers having a motif of 3-10-3.

Also provided are methods, compounds, and compositions useful for preventing, treating, and ameliorating diseases, disorders, and conditions. In certain embodiments, such HBV related diseases, disorders, and conditions are liver diseases. In certain embodiments, such liver diseases, disorders, and conditions includes jaundice, liver cancer, liver inflammation, liver fibrosis, inflammation, liver cirrhosis, liver failure, diffuse hepatocellular inflammatory disease, hemophagocytic syndrome, serum hepatitis, HBV viremia, and liver disease-related transplantation. In certain embodiments, such HBV related diseases, disorders, and conditions are hyperproliferative diseases, disorders, and conditions. In certain embodiments such hyperproliferative diseases, disorders, and conditions include cancer as well as associated malignancies and metastases. In certain embodiments, such cancers include liver cancer and hepatocellular cancer (HCC).

Such diseases, disorders, and conditions can have one or more risk factors, causes, or outcomes in common. Certain risk factors and causes for development of liver disease or a hyperproliferative disease include growing older; tobacco use; exposure to sunlight and ionizing radiation; contact with certain chemicals; infection with certain viruses and bacteria; certain hormone therapies; family history of cancer; alcohol use; and certain lifestyle choices including poor diet, lack of physical activity, and/or being overweight. Certain symptoms and outcomes associated with development of a liver disease or a hyperproliferative disease include but are not limited to: flu-like illness, weakness, aches, headache, fever, loss of appetite, diarrhea, jaundice, nausea and vomiting, pain over the liver area of the body, clay- or grey-colored stool, itching all over, and dark-colored urine.

In certain embodiments, methods of treatment include administering a HBV antisense compound to an individual in need thereof. In certain embodiments, methods of treatment include administering a HBV antisense oligonucleotide to an individual in need thereof.

### DETAILED DESCRIPTION

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference for the portions of the document discussed herein, as well as in their entirety.

### DEFINITIONS

Unless specific definitions are provided, the nomenclature utilized in connection with, and the procedures and techniques of, analytical chemistry, synthetic organic chemistry, and medicinal and pharmaceutical chemistry described herein are those well known and commonly used in the art. Standard techniques may be used for chemical synthesis, and chemical analysis. Where permitted, all patents, applications, published applications and other publications, GENBANK Accession Numbers and associated sequence information obtainable through databases such as National Center for Biotechnology Information (NCBI) and other data referred to throughout in the disclosure herein are incorporated by reference for the portions of the document discussed herein, as well as in their entirety.

Unless otherwise indicated, the following terms have the following meanings:

“3′ target site” refers to the nucleotide of a target nucleic acid which is complementary to the 3′-most nucleotide of a particular antisense compound.

“5′ target site” refers to the nucleotide of a target nucleic acid which is complementary to the 5′-most nucleotide of a particular antisense compound.

“5-methylcytosine” means a cytosine modified with a methyl group attached to the 5 position. A 5-methylcytosine is a modified nucleobase.

“About” means within ±7% of a value. For example, if it is stated, “the compounds affected at least about 70% inhibition of HBV”, it is implied that the HBV levels are inhibited within a range of 63% and 77%.

“Acceptable safety profile” means a pattern of side effects that is within clinically acceptable limits.

“Active pharmaceutical agent” means the substance or substances in a pharmaceutical composition that provide a therapeutic benefit when administered to an individual. For example, in certain embodiments an antisense oligonucleotide targeted to HBV is an active pharmaceutical agent.

“Active target region” means a target region to which one or more active antisense compounds is targeted. “Active antisense compounds” means antisense compounds that reduce target nucleic acid levels or protein levels.

“Acute hepatitis B infection” results when a person exposed to the hepatitis B virus begins to develop the signs and symptoms of viral hepatitis. This period of time, called the incubation period, is an average of 90 days, but could be as short as 45 days or as long as 6 months. For most people this infection will cause mild to moderate discomfort but will go away by itself because of the body's immune response succeeds in fighting the virus. However, some people, particularly those with compromised immune systems, such as persons suffering from AIDS, undergoing chemotherapy, taking immunosuppressant drugs, or taking steroids, have very serious problems as a result of the acute HBV infection, and go on to more severe conditions such as fulminant liver failure.

“Administered concomitantly” refers to the co-administration of two agents in any manner in which the pharmacological effects of both are manifest in the patient at the same time. Concomitant administration does not require that both agents be administered in a single pharmaceutical composition, in the same dosage form, or by the same route of administration. The effects of both agents need not manifest themselves at the same time. The effects need only be overlapping for a period of time and need not be coextensive.

“Administering” means providing a pharmaceutical agent to an individual, and includes, but is not limited to administering by a medical professional and self-administering.

“Agent” means an active substance that can provide a therapeutic benefit when administered to an animal. “First Agent” means a therapeutic compound described herein. For example, a first agent can be an antisense oligonucleotide targeting HBV. “Second agent” means a second therapeutic compound described herein (e.g. a second antisense oligonucleotide targeting HBV) and/or a non-HBV therapeutic compound.

“Amelioration” refers to a lessening of at least one indicator of the severity of a condition or disease. The severity of indicators may be determined by subjective or objective measures which are known to those skilled in the art.

“Animal” refers to a human or non-human animal, including, but not limited to, mice, rats, rabbits, dogs, cats, pigs, and non-human primates, including, but not limited to, monkeys and chimpanzees.

“Antibody” refers to a molecule characterized by reacting specifically with an antigen in some way, where the antibody and the antigen are each defined in terms of the other. Antibody may refer to a complete antibody molecule or any fragment or region thereof, such as the heavy chain, the light chain, F_{ab} region, and F_{c} region.

“Antisense activity” means any detectable or measurable activity attributable to the hybridization of an antisense compound to its target nucleic acid. In certain embodiments, antisense activity is a decrease in the amount or expression of a target nucleic acid or protein encoded by such target nucleic acid.

“Antisense compound” means an oligomeric compound that is capable of undergoing hybridization to a target nucleic acid through hydrogen bonding. Examples of antisense compounds include single-stranded and double-stranded compounds, such as, antisense oligonucleotides, siRNAs, shRNAs, snoRNAs, miRNAs, and satellite repeats.

“Antisense inhibition” means reduction of target nucleic acid levels in the presence of an antisense compound complementary to a target nucleic acid compared to target nucleic acid levels in the absence of the antisense compound.

“Antisense mechanisms” are all those mechanisms involving hybridization of a compound with target nucleic acid, wherein the outcome or effect of the hybridization is either target degradation or target occupancy with concomitant stalling of the cellular machinery involving, for example, transcription or splicing.

“Antisense oligonucleotide” means a single-stranded oligonucleotide having a nucleobase sequence that permits hybridization to a corresponding region or segment of a target nucleic acid.

“Base complementarity” refers to the capacity for the precise base pairing of nucleobases of an antisense oligonucleotide with corresponding nucleobases in a target nucleic acid (i.e., hybridization), and is mediated by Watson-Crick, Hoogsteen or reversed Hoogsteen hydrogen binding between corresponding nucleobases.

“Bicyclic sugar” means a furanose ring modified by the bridging of two non-geminal carbon atoms. A bicyclic sugar is a modified sugar. “Body weight” refers to an animal's whole body weight, inclusive of all tissues including adipose tissue.

“Body weight” refers to an animal's whole body weight, inclusive of all tissues including adipose tissue.

“Cap structure” or “terminal cap moiety” means chemical modifications, which have been incorporated at either terminus of an antisense compound.

“Chemically distinct region” refers to a region of an antisense compound that is in some way chemically different than another region of the same antisense compound. For example, a region having 4′-CH₂—O-2′ nucleotides is chemically distinct from a region having nucleotides without 4′-CH₂—O-2′ modifications.

“Chimeric antisense compounds” means antisense compounds that have at least 2 chemically distinct regions, a first region having at least one subunit and a second region having a plurality of subunits.

“Chronic hepatitis B infection” occurs when a person initially suffers from an acute infection but is then unable to fight off the infection. Whether the disease becomes chronic or completely resolves depends mostly on the age of the infected person. About 90% of infants infected at birth will progress to chronic disease. However, as a person ages, the risk of chronic infection decreases such that between 20%-50% of children and less than 10% of older children or adults will progress from acute to chronic infection. Chronic HBV infections are the primary treatment goal for embodiments of the present invention, although ASO compositions of the present invention are also capable of treating HBV-related conditions, such as inflammation, fibrosis, cirrhosis, liver cancer, serum hepatitis, and more.

“Co-administration” means administration of two or more pharmaceutical agents to an individual. The two or more pharmaceutical agents may be in a single pharmaceutical composition, or may be in separate pharmaceutical compositions. Each of the two or more pharmaceutical agents may be administered through the same or different routes of administration. Co-administration encompasses administration in parallel or sequentially.

“Complementarity” means the capacity for pairing between nucleobases of a first nucleic acid and a second nucleic acid.

“Comply” means the adherence with a recommended therapy by an individual.

“Comprise,” “comprises” and “comprising” will be understood to imply the inclusion of a stated step or element or group of steps or elements but not the exclusion of any other step or element or group of steps or elements.

“Contiguous nucleobases” means nucleobases immediately adjacent to each other.

“Cure” means a method or course that restores health or a prescribed treatment for an illness.

“Deoxyribonucleotide” means a nucleotide having a hydrogen at the 2′ position of the sugar portion of the nucleotide. Deoxyribonucleotides may be modified with any of a variety of substituents.

“Designing” or “Designed to” refer to the process of designing an oligomeric compound that specifically hybridizes with a selected nucleic acid molecule.

“Diluent” means an ingredient in a composition that lacks pharmacological activity, but is pharmaceutically necessary or desirable. For example, in drugs that are injected, the diluent may be a liquid, e.g. saline solution.

“Dosage unit” means a form in which a pharmaceutical agent is provided, e.g. pill, tablet, or other dosage unit known in the art.

“Dose” means a specified quantity of a pharmaceutical agent provided in a single administration, or in a specified time period. In certain embodiments, a dose may be administered in two or more boluses, tablets, or injections. For example, in certain embodiments, where subcutaneous administration is desired, the desired dose requires a volume not easily accommodated by a single injection. In such embodiments, two or more injections may be used to achieve the desired dose. In certain embodiments, a dose may be administered in two or more injections to minimize injection site reaction in an individual. In other embodiments, the pharmaceutical agent is administered by infusion over an extended period of time or continuously. Doses may be stated as the amount of pharmaceutical agent per hour, day, week or month.

“Dosing regimen” is a combination of doses designed to achieve one or more desired effects.

“Duration” means the period of time during which an activity or event continues. In certain embodiments, the duration of treatment is the period of time during which doses of a pharmaceutical agent are administered.

“Effective amount” in the context of modulating an activity or of treating or preventing a condition means the administration of that amount of active ingredient to a subject in need of such modulation, treatment or prophylaxis, either in a single dose or as part of a series, that is effective for modulation of that effect, or for treatment or prophylaxis or improvement of that condition. The effective amount will vary depending upon the health and physical condition of the subject to be treated, the taxonomic group of subjects to be treated, the formulation of the composition, the assessment of the medical situation, and other relevant factors.

“Efficacy” means the ability to produce a desired effect.

“Expression” includes all the functions by which a gene's coded information is converted into structures present and operating in a cell. Such structures include, but are not limited to the products of transcription and translation.

“Fully complementary” or “100% complementary” means each nucleobase of a first nucleic acid has a complementary nucleobase in a second nucleic acid. In certain embodiments, a first nucleic acid is an antisense compound and a target nucleic acid is a second nucleic acid.

“Fully modified motif” refers to an antisense compound comprising a contiguous sequence of nucleosides wherein essentially each nucleoside is a sugar modified nucleoside having uniform modification.

“Gapmer” means a chimeric antisense compound in which an internal region having a plurality of nucleosides that support RNase H cleavage is positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.”

“Gap-widened” means an antisense compound having a gap segment of 12 or more contiguous 2′-deoxyribonucleotides positioned between 5′ and 3′ wing segments having from one to six nucleotides having modified sugar moieties.

“HBV” means mammalian hepatitis B virus, including human hepatitis B virus. The term encompasses geographical genotypes of hepatitis B virus, particularly human hepatitis B virus, as well as variant strains of geographical genotypes of hepatitis B virus.

“HBV” means mammalian hepatitis B virus, including human hepatitis B virus. The term encompasses geographical genotypes of hepatitis B virus, particularly human hepatitis B virus, as well as variant strains of geographical genotypes of hepatitis B virus.

“HBV antigen” means any hepatitis B virus antigen or protein, including core proteins such as “hepatitis B core antigen” or “HBcAG” and “hepatitis B E antigen” or “HBeAG” and envelope proteins such as “HBV surface antigen”, or “HBsAg” or “HBsAG”.

“Hepatitis B E antigen” or “HBeAg” or “HBeAG” is a secreted, non-particulate form of HBV core protein. HBV antigens HBeAg and HBcAg share primary amino acid sequences, so show cross-reactivity at the T cell level. HBeAg is not required for viral assembly or replication, although studies suggest they may be required for establishment of chronic infection. Neonatal infection with HBeAg-negative mutant often results in fulminant acute rather than chronic HBV infection (Terezawa et al (1991) Pediatr. Res. 29:5), whereas infection of young woodchucks with WHeAg-negative mutant results in a much lower rate of chronic WHV infection (Cote et al (2000) Hepatology 31:190). HBeAg may possibly function as a toleragen by inactivating core specific T cells through deletion or clonal anergy (Milich et al (1998) J. Immunol. 160:8102). There is a positive correlation between reduction of HBV viral load and antigens, and a decrease of expression, by T cells, of the inhibitory receptor programmed death-1 (PD-1; also known as PDCD1), a negative regulator of activated T cells, upon antiviral therapy and HBeAg seroconversion (Evans et al (2008) Hepatology 48:759).

“HBV mRNA” means any messenger RNA expressed by hepatitis B virus.

“HBV nucleic acid” or ‘HBV DNA” means any nucleic acid encoding HBV. For example, in certain embodiments, a HBV nucleic acid includes, without limitation, any viral DNA sequence encoding a HBV genome or portion thereof, any RNA sequence transcribed from a viral DNA including any mRNA sequence encoding a HBV protein.

“HBV protein” means any protein secreted by hepatitis B virus The term encompasses various HBV antigens, including core proteins such as “Hepatitis E antigen”, “HBeAg” or “HBeAG” and envelope proteins such as “HBV surface antigen”, “HBsAg” or “HBsAG”.

“HBV surface antigen”, or “HBsAg”, or “HBsAG” is the envelope protein of infectious HBV viral particles but is also secreted as a non-infectious particle with serum levels 1000-fold higher than HBV viral particles. The serum levels of HBsAg in an infected person or animal can be as high as 1000 μg/mL (Kann and Gehrlich (1998) Topley & Wilson's Microbiology and Microbial Infections, 9^{th} ed. 745). In acute HBV infections, the half-life of HBsAg in the serum, or serum t %, is 8.3 days (Chulanov et al (2003) J. Med. Virol. 69: 313). Internalization of HBsAg by myeloid dendritic cells inhibits up-regulation of co-stimulatory molecules (i.e. B7) and inhibits T cell stimulatory capacity (den Brouw et al (2008) Immunology 126:280), and dendritic cells from chronically infected patients also show deficits in expression of co-stimulatory molecules, secretion of IL-12, and stimulation of T cells in the presence of HBsAg (Zheng et al (2004) J. Viral Hepatitis 11:217). HBsAg specific CD8 cells from CHB patients show altered tetramer binding. These CD8 cells are not anergic but may have TCR topology that confers partial tolerance or ignorance (Reignat et al (2002) J. Exp. Med. 195:1089). Moreover, reduction in serum HBsAg>1 log at week 24 has a high predictive value (92%) for sustained virological response (SVR—defined as nondetectable HBV DNA by PCR at 1 year after treatment) during Peg-IFNα2a therapy (Moucari et al (2009) Hepatology 49:1151).

“Hepatitis B-related condition” or “HBV-related condition” means any disease, biological condition, medical condition, or event which is exacerbated, caused by, related to, associated with, or traceable to a hepatitis B infection, exposure, or illness. The term hepatitis B-related condition includes chronic HBV infection, inflammation, fibrosis, cirrhosis, liver cancer, serum hepatitis, jaundice, liver cancer, liver inflammation, liver fibrosis, liver cirrhosis, liver failure, diffuse hepatocellular inflammatory disease, hemophagocytic syndrome, serum hepatitis, HBV viremia, liver disease related to transplantation, and conditions having symptoms which may include any or all of the following: flu-like illness, weakness, aches, headache, fever, loss of appetite, diarrhea, nausea and vomiting, pain over the liver area of the body, clay- or grey-colored stool, itching all over, and dark-colored urine, when coupled with a positive test for presence of a hepatitis B virus, a hepatitis B viral antigen, or a positive test for the presence of an antibody specific for a hepatitis B viral antigen.

“Hybridization” means the annealing of complementary nucleic acid molecules. In certain embodiments, complementary nucleic acid molecules include, but are not limited to, an antisense compound and a nucleic acid target. In certain embodiments, complementary nucleic acid molecules include, but are not limited to, an antisense oligonucleotide and a nucleic acid target.

“Identifying an animal having an HBV infection” means identifying an animal having been diagnosed with an HBV; or, identifying an animal having any symptom of an HBV infection including, but not limited to chronic HBV infection, inflammation, fibrosis, cirrhosis, liver cancer, serum hepatitis, jaundice, liver cancer, liver inflammation, liver fibrosis, liver cirrhosis, liver failure, diffuse hepatocellular inflammatory disease, hemophagocytic syndrome, serum hepatitis, HBV viremia, liver disease related to transplantation, and conditions having symptoms which may include any or all of the following: flu-like illness, weakness, aches, headache, fever, loss of appetite, diarrhea, nausea and vomiting, pain over the liver area of the body, clay- or grey-colored stool, itching all over, and dark-colored urine, when coupled with a positive test for presence of a hepatitis B virus, a hepatitis B viral antigen, or a positive test for the presence of an antibody specific for a hepatitis B viral antigen, when coupled with a positive test for presence of a hepatitis B virus, a hepatitis B viral antigen, or a positive test for the presence of an antibody specific for a hepatitis B viral antigen.

“Immediately adjacent” means there are no intervening elements between the immediately adjacent elements.

“Individual” means a human or non-human animal selected for treatment or therapy.

“Individual compliance” means adherence to a recommended or prescribed therapy by an individual.

“Induce”, “inhibit”, “potentiate”, “elevate”, “increase”, “decrease” or the like, e.g., which denote quantitative differences between two states, refer to at least statistically significant differences between the two states. For example, “an amount effective to inhibit the activity or expression of HBV” means that the level of activity or expression of HBV in a treated sample will differ statistically significantly from the level of HBV activity or expression in untreated cells. Such terms are applied to, for example, levels of expression, and levels of activity.

“Inhibiting HBV” means reducing the level or expression of an HBV mRNA, DNA and/or protein. In certain embodiments, HBV is inhibited in the presence of an antisense compound targeting HBV, including an antisense oligonucleotide targeting HBV, as compared to expression of HBV mRNA, DNA and/or protein levels in the absence of a HBV antisense compound, such as an antisense oligonucleotide.

“Inhibiting the expression or activity” refers to a reduction, blockade of the expression or activity and does not necessarily indicate a total elimination of expression or activity.

“Injection site reaction” means inflammation or abnormal redness of skin at a site of injection in an individual.

“Internucleoside linkage” refers to the chemical bond between nucleosides.

“Intraperitoneal administration” means administration through infusion or injection into the peritoneum.

“Intravenous administration” means administration into a vein.

“Lengthened” antisense oligonucleotides are those that have one or more additional nucleosides relative to an antisense oligonucleotide disclosed herein.

“Linked deoxynucleoside” means a nucleic acid base (A, G, C, T, U) substituted by deoxyribose linked by a phosphate ester to form a nucleotide.

“Linked nucleosides” means adjacent nucleosides linked together by an internucleoside linkage.

“Locked nucleic acid” or “LNA” or “LNA nucleosides” means nucleic acid monomers having a bridge connecting two carbon atoms between the 4′ and 2′ position of the nucleoside sugar unit, thereby forming a bicyclic sugar. Examples of such bicyclic sugar include, but are not limited to A) α-L-Methyleneoxy (4′-CH₂—O-2′) LNA, (B) β-D-Methyleneoxy (4′-CH₂—O-2′) LNA, (C) Ethyleneoxy (4′-(CH₂)₂—O-2′) LNA, (D) Aminooxy (4′-CH₂—O—N(R)-2′) LNA and (E) Oxyamino (4′-CH₂—N(R)—O-2′) LNA, as depicted below.

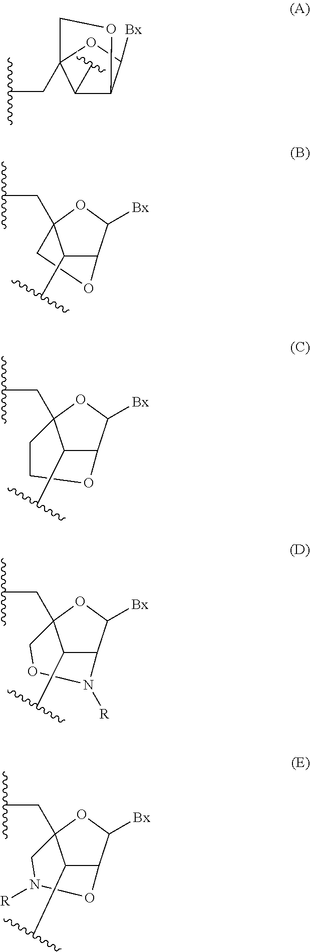

As used herein, LNA compounds include, but are not limited to, compounds having at least one bridge between the 4′ and the 2′ position of the sugar wherein each of the bridges independently comprises 1 or from 2 to 4 linked groups independently selected from —[C(R₁)(R₂)]ₙ—, —C(R₁)═C(R₂)—, —C(R₁)═N—, —C(═NR₁)—, —C(═O)—, —C(═S)—, —O—, —Si(R₁)₂—, —S(═O)ₓ— and —N(R₁)—; wherein: x is 0, 1, or 2; n is 1, 2, 3, or 4; each R₁ and R₂ is, independently, H, a protecting group, hydroxyl, C₁-C₁₂ alkyl, substituted C₁-C₁₂ alkyl, C₂-C₁₂ alkenyl, substituted C₁-C₁₂ alkenyl, C₂-C₁₂ alkynyl, substituted C₁-C₁₂ alkynyl, C₅-C₂₀ aryl, substituted C₅-C₂₀ aryl, a heterocycle radical, a substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

Examples of 4′-2′ bridging groups encompassed within the definition of LNA include, but are not limited to one of formulae: —[C(R₁)(R₂)]ₙ—, —[C(R₁)(R₂)]ₙ—O—, —C(R₁R₂)—N(R₁)—O— or —C(R₁R₂)—O—N(R₁)—. Furthermore, other bridging groups encompassed with the definition of LNA are 4′-CH₂-2′,4′-(CH₂)₂-2′,4′-(CH₂)₃-2′,4′-CH₂—O-2′,4′-(CH₂)₂—O-2′,4′-CH₂—O—N(R₁)-2′ and 4-CH₂—N(R₁)—O-2′-bridges, wherein each R₁ and R₂ is, independently, H, a protecting group or C₁-C₁₂ alkyl.

Also included within the definition of LNA according to the invention are LNAs in which the 2′-hydroxyl group of the ribosyl sugar ring is connected to the 4′ carbon atom of the sugar ring, thereby forming a methyleneoxy (4′-CH₂—O-2′) bridge to form the bicyclic sugar moiety. The bridge can also be a methylene (—CH₂—) group connecting the 2′ oxygen atom and the 4′ carbon atom, for which the term methyleneoxy (4′-CH₂—O-2′) LNA is used. Furthermore; in the case of the bicylic sugar moiety having an ethylene bridging group in this position, the term ethyleneoxy (4′-CH₂CH₂—O-2′) LNA is used. α-L-methyleneoxy (4′-CH₂—O-2′), an isomer of methyleneoxy (4′-CH₂—O-2′) LNA is also encompassed within the definition of LNA, as used herein.

“Mismatch” or “non-complementary nucleobase” refers to the case when a nucleobase of a first nucleic acid is not capable of pairing with the corresponding nucleobase of a second or target nucleic acid.

“Mismatch” or “non-complementary nucleobase” refers to the case when a nucleobase of a first nucleic acid is not capable of pairing with the corresponding nucleobase of a second or target nucleic acid.

“Modified internucleoside linkage” refers to a substitution or any change from a naturally occurring internucleoside bond (i.e. a phosphodiester internucleoside bond).

“Modified nucleobase” means any nucleobase other than adenine, cytosine, guanine, thymidine, or uracil. An “unmodified nucleobase” means the purine bases adenine (A) and guanine (G), and the pyrimidine bases thymine (T), cytosine (C) and uracil (U).

“Modified nucleoside” means a nucleoside having, independently, a modified sugar moiety and/or modified nucleobase.

“Modified nucleotide” means a nucleotide having, independently, a modified sugar moiety, modified internucleoside linkage, or modified nucleobase.

“Modified oligonucleotide” means an oligonucleotide comprising at least one modified internucleoside linkage, a modified sugar, and/or a modified nucleobase.

“Modified sugar” means substitution and/or any change from a natural sugar moiety.

“Monomer” refers to a single unit of an oligomer. Monomers include, but are not limited to, nucleosides and nucleotides, whether naturally occurring or modified.

“Motif” means the pattern of unmodified and modified nucleosides in an antisense compound.

“Natural sugar moiety” means a sugar moiety found in DNA (2′-H) or RNA (2′-OH).

“Naturally occurring internucleoside linkage” means a 3′ to 5′ phosphodiester linkage.

“Non-complementary nucleobase” refers to a pair of nucleobases that do not form hydrogen bonds with one another or otherwise support hybridization.

“Nucleic acid” refers to molecules composed of monomeric nucleotides. A nucleic acid includes, but is not limited to, ribonucleic acids (RNA), deoxyribonucleic acids (DNA), single-stranded nucleic acids, double-stranded nucleic acids, small interfering ribonucleic acids (siRNA), and microRNAs (miRNA).

“Nucleobase” means a heterocyclic moiety capable of pairing with a base of another nucleic acid.

“Nucleobase complementarity” refers to a nucleobase that is capable of base pairing with another nucleobase. For example, in DNA, adenine (A) is complementary to thymine (T). For example, in RNA, adenine (A) is complementary to uracil (U). In certain embodiments, complementary nucleobase refers to a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair.

“Nucleobase sequence” means the order of contiguous nucleobases independent of any sugar, linkage, and/or nucleobase modification.

“Nucleoside” means a nucleobase linked to a sugar.

“Nucleoside mimetic” includes those structures used to replace the sugar or the sugar and the base and not necessarily the linkage at one or more positions of an oligomeric compound such as for example nucleoside mimetics having morpholino, cyclohexenyl, cyclohexyl, tetrahydropyranyl, bicyclo or tricyclo sugar mimetics, e.g., non furanose sugar units. Nucleotide mimetic includes those structures used to replace the nucleoside and the linkage at one or more positions of an oligomeric compound such as for example peptide nucleic acids or morpholinos (morpholinos linked by —N(H)—C(═O)—O— or other non-phosphodiester linkage). Sugar surrogate overlaps with the slightly broader term nucleoside mimetic but is intended to indicate replacement of the sugar unit (furanose ring) only. The tetrahydropyranyl rings provided herein are illustrative of an example of a sugar surrogate wherein the furanose sugar group has been replaced with a tetrahydropyranyl ring system. “Mimetic” refers to groups that are substituted for a sugar, a nucleobase, and/or internucleoside linkage. Generally, a mimetic is used in place of the sugar or sugar-internucleoside linkage combination, and the nucleobase is maintained for hybridization to a selected target.

“Nucleotide” means a nucleoside having a phosphate group covalently linked to the sugar portion of the nucleoside.

“Off-target effect” refers to an unwanted or deleterious biological effect associated with modulation of RNA or protein expression of a gene other than the intended target nucleic acid.

“Oligomeric compound” means a polymer of linked monomeric subunits which is capable of hybridizing to at least a region of a nucleic acid molecule.

“Oligonucleoside” means an oligonucleotide in which the internucleoside linkages do not contain a phosphorus atom.

“Oligonucleotide” means a polymer of linked nucleosides each of which can be modified or unmodified, independent one from another.

“Parenteral administration” means administration through injection (e.g., bolus injection) or infusion. Parenteral administration includes subcutaneous administration, intravenous administration, intramuscular administration, intraarterial administration, intraperitoneal administration, or intracranial administration, e.g., intrathecal or intracerebroventricular administration.

“Peptide” means a molecule formed by linking at least two amino acids by amide bonds. Without limitation, as used herein, “peptide” refers to polypeptides and proteins.

“Pharmaceutically acceptable carrier” means a medium or diluent that does not interfere with the structure of the oligonucleotide. Certain such carriers enable pharmaceutical compositions to be formulated as, for example, tablets, pills, dragees, capsules, liquids, gels, syrups, slurries, suspension and lozenges for the oral ingestion by a subject.

“Pharmaceutically acceptable derivative” encompasses pharmaceutically acceptable salts, conjugates, prodrugs or isomers of the compounds described herein.

“Pharmaceutically acceptable salts” means physiologically and pharmaceutically acceptable salts of antisense compounds, i.e., salts that retain the desired biological activity of the parent oligonucleotide and do not impart undesired toxicological effects thereto.

“Pharmaceutical agent” means a substance that provides a therapeutic benefit when administered to an individual. For example, in certain embodiments, an antisense oligonucleotide targeted to HBV is a pharmaceutical agent.

“Pharmaceutical composition” means a mixture of substances suitable for administering to a subject. For example, a pharmaceutical composition may comprise an antisense oligonucleotide and a sterile aqueous solution. In certain embodiments, a pharmaceutical composition shows activity in free uptake assay in certain cell lines.

“Phosphorothioate linkage” means a linkage between nucleosides where the phosphodiester bond is modified by replacing one of the non-bridging oxygen atoms with a sulfur atom. A phosphorothioate linkage is a modified internucleoside linkage.

“Portion” means a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an antisense compound.

“Prevention” or “preventing” refers to delaying or forestalling the onset or development of a condition or disease for a period of time from hours to days, preferably weeks to months.

“Prodrug” means a therapeutic agent that is prepared in an inactive form that is converted to an active form (i.e., drug) within the body or cells thereof by the action of endogenous enzymes or other chemicals and/or conditions.

“Prophylactically effective amount” refers to an amount of a pharmaceutical agent that provides a prophylactic or preventative benefit to an animal.

“Recommended therapy” means a therapeutic regimen recommended by a medical professional for the treatment, amelioration, or prevention of a disease.

“Region” is defined as a portion of the target nucleic acid having at least one identifiable structure, function, or characteristic.

“Ribonucleotide” means a nucleotide having a hydroxy at the 2′ position of the sugar portion of the nucleotide. Ribonucleotides may be modified with any of a variety of substituents.

“Salts” mean a physiologically and pharmaceutically acceptable salts of antisense compounds, i.e., salts that retain the desired biological activity of the parent oligonucleotide and do not impart undesired toxicological effects thereto.

“Segments” are defined as smaller or sub-portions of regions within a target nucleic acid.

“Seroconversion” is defined as serum HBeAg absence plus serum HBeAb presence, if monitoring HBeAg as the determinant for seroconversion, or defined as serum HBsAg absence, if monitoring HBsAg as the determinant for seroconversion, as determined by currently available detection limits of commercial ELISA systems.

“Shortened” or “truncated” versions of antisense oligonucleotides taught herein have one, two or more nucleosides deleted.

“Side effects” means physiological responses attributable to a treatment other than desired effects. In certain embodiments, side effects include, without limitation, injection site reactions, liver function test abnormalities, renal function abnormalities, liver toxicity, renal toxicity, central nervous system abnormalities, and myopathies. For example, increased aminotransferase levels in serum may indicate liver toxicity or liver function abnormality. For example, increased bilirubin may indicate liver toxicity or liver function abnormality.

“Sites,” as used herein, are defined as unique nucleobase positions within a target nucleic acid.

“Slows progression” means decrease in the development of the said disease.

“Specifically hybridizable” refers to an antisense compound having a sufficient degree of complementarity between an antisense oligonucleotide and a target nucleic acid to induce a desired effect, while exhibiting minimal or no effects on non-target nucleic acids under conditions in which specific binding is desired, i.e., under physiological conditions in the case of in vivo assays and therapeutic treatments. “Statin” means an agent that inhibits the activity of HMG-CoA reductase.

“Stringent hybridization conditions” or “stringent conditions” refer to conditions under which an oligomeric compound will hybridize to its target sequence, but to a minimal number of other sequences.

“Subcutaneous administration” means administration just below the skin.

“Subject” means a human or non-human animal selected for treatment or therapy.

“Target” refers to a protein, the modulation of which is desired.

“Target gene” refers to a gene encoding a target.

“Targeting” means the process of design and selection of an antisense compound that will specifically hybridize to a target nucleic acid and induce a desired effect.

“Target nucleic acid,” “target RNA,” “target RNA transcript” and “nucleic acid target” all mean a nucleic acid capable of being targeted by antisense compounds.

“Target region” means a portion of a target nucleic acid to which one or more antisense compounds is targeted.

“Target segment” means the sequence of nucleotides of a target nucleic acid to which an antisense compound is targeted. “5′ target site” refers to the 5′-most nucleotide of a target segment. “3′ target site” refers to the 3′-most nucleotide of a target segment.

“Therapeutically effective amount” means an amount of a pharmaceutical agent that provides a therapeutic benefit to an individual.

“Treatment” refers to administering a composition described herein to effect an alteration or improvement of the disease or condition.

“Unmodified” nucleobases mean the purine bases adenine (A) and guanine (G), and the pyrimidine bases thymine (T), cytosine (C) and uracil (U).

“Unmodified nucleotide” means a nucleotide composed of naturally occurring nucleobases, sugar moieties, and internucleoside linkages. In certain embodiments, an unmodified nucleotide is an RNA nucleotide (i.e. β-D-ribonucleosides) or a DNA nucleotide (i.e. β-D-deoxyribonucleoside).

“Validated target segment” is defined as at least an 8-nucleobase portion (i.e. 8 consecutive nucleobases) of a target region to which an active oligomeric compound is targeted.

“Wing segment” means a plurality of nucleosides modified to impart to an oligonucleotide properties such as enhanced inhibitory activity, increased binding affinity for a target nucleic acid, or resistance to degradation by in vivo nucleases.

### CERTAIN EMBODIMENTS

In certain embodiments, the compounds provided herein are or comprise a modified oligonucleotide. In certain embodiments the compounds comprise a modified oligonucleotide and a conjugate as described herein. In certain embodiments, the modified oligonucleotide is a pharmaceutically acceptable derivative.

In certain embodiments, the compounds or compositions comprise a modified oligonucleotide 10 to 30 linked nucleosides in length targeted to HBV. The HBV target can have a sequence recited in SEQ ID NO: 1 or a portion thereof or a variant thereof. In certain embodiments, such compounds or modified oligonucleotides target one of the following nucleotide regions of HBV: CCTGCTGGTGGCTCCAGTTC (SEQ ID NO: 2); AGAGTCTAGACTCGTGGTGGACTTCTCTCAATTTTCTAGGGG (SEQ ID NO: 3); TGGATGTGTCTGCGGCGTTTTATCAT (SEQ ID NO: 4); CATCCTGCTGCTATGCCTCATCTTCTT (SEQ ID NO: 5); CAAGGTATGTTGCCCGT (SEQ ID NO: 6); TGTATTCCCATCCCATC (SEQ ID NO: 7); CCTATGGGAGTGGGCCTCAG (SEQ ID NO: 8); TGGCTCAGTTTACTAGTGC (SEQ ID NO: 9); GGGCTTTCCCCCACTGT (SEQ ID NO: 10); TCCTCTGCCGATCCATACTGCGGAACTCCT (SEQ ID NO: 11); CGCACCTCTCTTTACGCGG (SEQ ID NO: 12); GGAGTGTGGATTCGCAC (SEQ ID NO: 13); or GAAGAAGAACTCCCTCGCCT (SEQ ID NO: 14). In certain embodiments, such compounds or oligonucleotides have a gap segment of 10 or more linked deoxynucleosides. In certain embodiments, such gap segment is between two wing segments that independently have 1-5, 1-4, 1-3, 2-5, 2-4 or 2-3 linked modified nucleosides. In certain embodiments, one or more modified nucleosides in the wing segment have a modified sugar. In certain embodiments, the modified sugar is a bicyclic sugar. In certain embodiments, the modified nucleoside is an LNA nucleoside.

In certain embodiments, the compounds or compositions comprise a modified oligonucleotide consisting of 10 to 30 nucleosides having a nucleobase sequence comprising at least 8 contiguous nucleobases complementary to an equal length portion of SEQ ID NOs: 1-14.

In certain embodiments, the compounds or compositions comprise a modified oligonucleotide consisting of 10 to 30 linked nucleosides and having a nucleobase sequence comprising at least 8, 9, 10, 11, 12, 13, 14, 15, 16, 17, 18, 19, 20, 21, 22, 23, 24, 25, 26, 27, 28, 29 or 30 contiguous nucleobases complementary to an equal length portion of SEQ ID NOs: 1-14. In certain embodiments, such oligonucleotides have a gap segment of 10 or more linked deoxynucleosides. In certain embodiments, such gap segment is between two wing segments that independently have 1-5, 1-4, 1-3, 2-5, 2-4 or 2-3 linked modified nucleosides. In certain embodiments, one or more modified nucleosides in the wing segment have a modified sugar. In certain embodiments, the modified sugar is a bicyclic sugar. In certain embodiments, the modified nucleoside is an LNA nucleoside.

In certain embodiments, the compounds or compositions comprise a modified oligonucleotide consisting of 10 to 30 linked nucleosides and having a nucleobase sequence comprising at least 8, 9, 10, 11, 12, 13, 14, 15, 16, 17, 18, 19, or 20 contiguous nucleobases of any of SEQ ID NOs: 18-35. In certain embodiments, such oligonucleotides have a gap segment of 10 or more linked deoxynucleosides. In certain embodiments, such gap segment is between two wing segments that independently have 1-5, 1-4, 1-3, 2-5, 2-4 or 2-3 linked modified nucleosides. In certain embodiments, one or more modified nucleosides in the wing segment have a modified sugar. In certain embodiments, the modified sugar is a bicyclic sugar. In certain embodiments, the modified nucleoside is an LNA nucleoside.

In certain embodiments, the compounds or compositions of the invention can consist of 10 to 30 linked nucleosides and have a nucleobase sequence comprising at least 8, 9, 10, 11, 12, 13, 14, 15, 16, 17, 18, 19, or 20 contiguous nucleobases of any of SEQ ID NOs: 18-35.

In certain embodiments, the compounds or compositions comprise a salt of the modified oligonucleotide.

In certain embodiments, the compounds or compositions further comprise a pharmaceutically acceptable carrier or diluent.

In certain embodiments, the nucleobase sequence of the modified oligonucleotide is at least 70%, 75%, 80%, 85%, 90%, 95% or 100% complementary to any one of SEQ ID NO: 1-14 as measured over the entirety of the modified oligonucleotide.

In certain embodiments, the compound or modified oligonucleotide is single-stranded. In certain embodiments, the modified oligonucleotide consists of 8, 9, 10, 11, 12, 13, 14, 15, 16, 17, 18, 19, 20, 21, 22, 23, 24, 25, 26, 27, 28, 29 or 30 linked nucleosides. In certain embodiments, the modified oligonucleotide consists of 20 linked nucleosides. In certain embodiments, the modified oligonucleotide consists of 18 linked nucleosides. In certain embodiments, the modified oligonucleotide consists of 17 linked nucleosides. In certain embodiments, the modified oligonucleotide consists of 16 linked nucleosides. In certain embodiments, the modified oligonucleotide consists of 14 linked nucleosides.

In certain embodiments, at least one internucleoside linkage of the modified oligonucleotide is a modified internucleoside linkage. In certain embodiments, each internucleoside linkage is a phosphorothioate internucleoside linkage.

In certain embodiments, at least one nucleoside of the modified oligonucleotide comprises a modified sugar.

In certain embodiments, at least one modified sugar is a bicyclic sugar. In certain embodiments, at least one modified sugar the bicyclic sugar comprises a 4′-(CH₂)ₙ—O-2′ bridge, wherein n is 1 or 2. In certain embodiments, the bicyclic sugar comprises a 4′-CH₂—O-2′ bridge.

In certain embodiments, at least one nucleoside of said modified oligonucleotide comprises a modified nucleobase. In certain embodiments, the modified nucleobase is a 5-methylcytosine.

In certain embodiments, the modified oligonucleotide consists of a single-stranded modified oligonucleotide.

In certain embodiments, the modified oligonucleotide comprises: a) a gap segment consisting of linked deoxynucleosides; b) a 5′ wing segment consisting of linked nucleosides; and c) a 3′ wing segment consisting of linked nucleosides. The gap segment is positioned between the 5′ wing segment and the 3′ wing segment and each nucleoside of each wing segment comprises a modified sugar.

In certain embodiments, the modified oligonucleotide consists of 16 linked nucleosides, the gap segment consisting of ten linked deoxynucleosides, the 5′ wing segment consisting of three linked nucleosides, the 3′ wing segment consisting of three linked nucleosides, each nucleoside of each wing segment comprises a locked nucleic acid (LNA), each internucleoside linkage is a phosphorothioate linkage and each cytosine is a 5-methylcytosine.

In certain embodiments, the modified oligonucleotide consists of 14 linked nucleosides, the gap segment consisting of ten linked deoxynucleosides, the 5′ wing segment consisting of two linked nucleosides, the 3′ wing segment consisting of two linked nucleosides, each nucleoside of each wing segment comprises locked nucleic acid (LNA), each internucleoside linkage is a phosphorothioate linkage and each cytosine is a 5-methylcytosine.

In certain embodiments, the compounds or compositions comprise a modified oligonucleotide consisting of 16 linked nucleosides having a nucleobase sequence comprising at least 8 contiguous nucleobases complementary to an equal length portion of any of SEQ ID NO: 2-14, wherein the modified oligonucleotide comprises: a) a gap segment consisting of ten linked deoxynucleosides; b) a 5′ wing segment consisting of three linked nucleosides; and c) a 3′ wing segment consisting of three linked nucleosides. The gap segment is positioned between the 5′ wing segment and the 3′ wing segment, each nucleoside of each wing segment comprises a locked nucleic acid (LNA), each internucleoside linkage is a phosphorothioate linkage and each cytosine residue is a 5-methylcytosine.

In certain embodiments, the compounds or compositions comprise a modified oligonucleotide consisting of 14 linked nucleosides having a nucleobase sequence comprising at least 8 contiguous nucleobases complementary to an equal length portion of any of SEQ ID NO: 2-14, wherein the modified oligonucleotide comprises: a) a gap segment consisting of ten linked deoxynucleosides; b) a 5′ wing segment consisting of two linked nucleosides; and c) a 3′ wing segment consisting of two linked nucleosides. The gap segment is positioned between the 5′ wing segment and the 3′ wing segment, each nucleoside of each wing segment comprises a locked nucleic acid (LNA), each internucleoside linkage is a phosphorothioate linkage and each cytosine residue is a 5-methylcytosine.

Certain embodiments provide methods, compounds, and compositions for inhibiting HBV expression. In certain embodiments, the provided methods, compounds, and compositions inhibit HBV mRNA expression and/or DNA levels and or protein levels and/or antigen levels.

Another embodiment provides a method for treating a HBV-related diseases, disorders, and conditions in a mammal, the method comprising administering a therapeutically effective amount of any pharmaceutical composition as described above to a mammal in need thereof, so as to treat the HBV-related diseases, disorders, and condition. In related embodiments, the mammal is a human and the HBV-related disease, disorder, and condition is a hepatitis B virus infection from a human hepatitis B virus. More particularly, the human hepatitis B virus may be any of the human geographical genotypes: A (Northwest Europe, North America, Central America); B (Indonesia, China, Vietnam); C (East Asia, Korea, China, Japan, Polynesia, Vietnam); D (Mediterranean area, Middle East, India); E (Africa); F (Native Americans, Polynesia); G (United States, France); or H (Central America).

In certain embodiments, an antisense compound or oligonucleotide targeted to a HBV nucleic acid target the following nucleotide regions of SEQ ID NO 1: 258-274, 258-273, 259-272, 260-273, 414-429, 414-432, 414-430, 415-30, 417-432, 415-432, 415-428, 416-429, 416-426, 418-431, 602-617, 603-616, 639-654, 639-658, 639-655, 640-658, 640-653, 640-655, 641-654, 643-658, and 644-657. In certain embodiments, such oligonucleotides have a gap segment of 10 or more linked deoxynucleosides. In certain embodiments, such gap segment is between two wing segments that independently have 1-5, 1-4, 1-3, 2-5, 2-4 or 2-3 linked modified nucleosides. In certain embodiments, one or more modified nucleosides in the wing segment have a modified sugar. In certain embodiments, the modified sugar is a bicyclic sugar. In certain embodiments, the modified nucleoside is an LNA nucleoside.

In certain embodiments, antisense compounds or oligonucleotides target a region of a HBV nucleic acid. In certain embodiments, such compounds or oligonucleotides targeted to a region of a HBV nucleic acid have a contiguous nucleobase portion that is complementary to an equal length nucleobase portion of the region. For example, the portion can be at least an 8, 9, 10, 11, 12, 13, 14, 15, 16, 17, 18, 19 or 20 contiguous nucleobases portion complementary to an equal length portion of a region recited herein. In certain embodiments, such compounds or oligonucleotide target the following nucleotide regions of SEQ ID NO: 1, CCTGCTGGTGGCTCCAGTTC (SEQ ID NO: 2); AGAGTCTAGACTCGTGGTGGACTTCTCTCAATTTTCTAGGGG (SEQ ID NO: 3); TGGATGTGTCTGCGGCGTTTTATCAT (SEQ ID NO: 4); CATCCTGCTGCTATGCCTCATCTTCTT (SEQ ID NO: 5); CAAGGTATGTTGCCCGT (SEQ ID NO: 6); TGTATTCCCATCCCATC (SEQ ID NO: 7); CCTATGGGAGTGGGCCTCAG (SEQ ID NO: 8); TGGCTCAGTTTACTAGTGC (SEQ ID NO: 9); GGGCTTTCCCCCACTGT (SEQ ID NO: 10); TCCTCTGCCGATCCATACTGCGGAACTCCT (SEQ ID NO: 11); CGCACCTCTCTTTACGCGG (SEQ ID NO: 12); GGAGTGTGGATTCGCAC (SEQ ID NO: 13); or GAAGAAGAACTCCCTCGCCT (SEQ ID NO: 14). In certain embodiments, such oligonucleotides have a gap segment of 10 or more linked deoxynucleosides. In certain embodiments, such gap segment is between two wing segments that independently have 1-5, 1-4, 1-3, 2-5, 2-4 or 2-3 linked modified nucleosides. In certain embodiments, one or more modified nucleosides in the wing segment have a modified sugar. In certain embodiments, the modified sugar is a bicyclic sugar. In certain embodiments, the modified nucleoside is an LNA nucleoside.

In certain embodiments, the compounds as described herein are efficacious by virtue of having at least one of an in vitro IC₅₀ of less than 20 μM, less than 15 μM, less than 10 μM, less than 6 μM, less than 5 μM, less than 4 μM, less than 3 μM, less than 2 less than 1.9 μM, less than 1.8 μM, less than 1.75 μM, when delivered to HepG2.2.15 cells.

In certain embodiments, inhibition is measured with primer probe set RTS3370, wherein the compounds are efficacious by virtue of having at least one of an in vitro IC₅₀ of less than 20 μM, less than 15 μM, less than 10 μM, less than 6 μM, less than 5 μM, less than 4 μM, less than 3 μM, less than 2 μM, less than 1.9 μM, less than 1.8 μM, less than 1.75 μM, when delivered to HepAD38 cells In certain embodiments, the compounds as described herein are efficacious by virtue of having at least one of an in vitro IC₅₀ of less than 20 μM, less than 15 μM, less than 10 μM, less than 6 μM, less than 5 μM, less than 4 μM, less than 3 μM, less than 2 μM, less than 1.9 μM, less than 1.8 μM, less than 1.7 μM, less than 1.6 μM, less than 1.5 μM, less than 1.4 μM, less than 1.3 μM, less than 1.25 μM, when delivered to HepAD38 cells and measured with primer probe set RTS3372, as described herein.

In certain embodiments, the compounds as described herein are efficacious by virtue of having at least one of an in vitro IC₅₀ of less than 20 μM, less than 15 μM, less than 10 μM, less than 6 μM, less than 5 μM, less than 4 μM, less than 3 μM, less than 2.9 μM, when delivered to HepAD38 cells and measured with primer probe set RTS3373MGB, as described herein.

In certain embodiments, such compounds include compounds comprising the nucleobase sequence of any one of SEQ ID NOs: 18-35.

In certain embodiments, the following nucleotide regions of SEQ ID NO: 1 are targeted by antisense compounds or oligonucleotides that have an in vitro IC₅₀ of less than 20 μM when delivered to HepG2.2.15 cells and measured with primer probe set RTS3370, as described herein: 258-273, 414-429, 417-432, 602-617, 639-655, 643-658.

In certain embodiments, the following nucleotide regions of SEQ ID NO: 1 are targeted by antisense compounds or oligonucleotides that have an in vitro IC₅₀ of less than 10 μM when delivered to HepG2.2.15 cells and measured with primer probe set RTS3370, as described herein: 258-274, 414-429, 415-432, 418-431, 602-616, 640-655, and 643-658.

In certain embodiments, the following nucleotide regions of SEQ ID NO: 1 are targeted by antisense compounds or oligonucleotides that have an in vitro IC₅₀ of less than 5 μM when delivered to HepG2.2.15 cells and measured with primer probe set RTS3370, as described herein: 258-274, 414-429, 415-432, and 602-617.

In certain embodiments, the following nucleotide regions of SEQ ID NO: 1 are targeted by antisense compounds or oligonucleotides that have an in vitro IC₅₀ of less than 20 μM when delivered to HepAD38 cells and measured with primer probe set RTS3372, as described herein: 258-274, 414-430, 417-432, 602-617, 639-655, and 643-658.

In certain embodiments, the following nucleotide regions of SEQ ID NO: 1 are targeted by antisense compounds or oligonucleotides that have an in vitro IC₅₀ of less than 10 μM when delivered to HepAD38 cells and measured with primer probe set RTS3372, as described herein: 258-274, 414-429, 415-429, 417-432, 602-617, 639-655, and 643-658.

In certain embodiments, the following nucleotide regions of SEQ ID NO: 1 are targeted by antisense compounds or oligonucleotides that have an in vitro IC₅₀ of less than 5 μM when delivered to HepAD38 cells and measured with primer probe set RTS3372, as described herein: 414-429, 417-432, 602-617, and 643-658.

In certain embodiments, the following nucleotide regions of SEQ ID NO: 1 are targeted by antisense compounds or oligonucleotides that have an in vitro IC₅₀ of less than 20 μM when delivered to HepAD38 cells and measured with primer probe set RTS3373MGB, as described herein: 258-274, 260-273, 414-430, 417-432, 602-617, 639-654, and 643-658.

In certain embodiments, the following nucleotide regions of SEQ ID NO: 1 are targeted by antisense compounds or oligonucleotides that have an in vitro IC₅₀ of less than 10 μM when delivered to HepAD38 cells and measured with primer probe set RTS3373MGB, as described herein: 259-274, 260-273, 414-429, 415-432, 602-617, and 643-658.

In certain embodiments, the following nucleotide regions of SEQ ID NO: 1 are targeted by antisense compounds or oligonucleotides that have an in vitro IC₅₀ of less than 5 μM when delivered to HepAD38 cells and measured with primer probe set RTS3373MGB, as described herein: 415-428, 417-432, and 602-617.

Certain embodiments provide methods of treating HBV related disease, disorder, or condition in an animal, comprising administering to an animal in need thereof a compound or composition described herein. In certain embodiments, the compound or composition comprises a modified oligonucleotide consisting of 10 to 30 linked nucleosides and having a nucleobase sequence comprising at least 10 contiguous nucleobases of any of the nucleobase sequences of SEQ ID NOs: 18-35.

Certain embodiments provide a method of reducing HBV expression in an animal comprising administering to the animal a compound or composition described herein. In certain embodiments, the compound or composition comprises a modified oligonucleotide 10 to 30 linked nucleosides in length targeted to HBV and having a nucleobase sequence comprising at least 10 contiguous nucleobases of any of the nucleobase sequences of SEQ ID NOs: 18-35.

Certain embodiments provide a method of preventing, ameliorating or treating an HBV-related disease, disorder or condition in an animal comprising administering to the animal a compound or composition described herein. In certain embodiments, the compound or composition comprises a modified oligonucleotide 10 to 30 linked nucleosides in length targeted to HBV. In certain embodiments, the modified oligonucleotide has a nucleobase sequence comprising at least 10 contiguous nucleobases of any of the nucleobase sequences of SEQ ID NOs: 18-35. Examples of HBV-related diseases, disorders or conditions include, but are not limited to chronic HBV infection, jaundice, liver cancer, liver inflammation, liver fibrosis, liver cirrhosis, liver failure, diffuse hepatocellular inflammatory disease, hemophagocytic syndrome, serum hepatitis, HBV viremia, and conditions having symptoms which may include any or all of the following: flu-like illness, weakness, aches, headache, fever, loss of appetite, diarrhea, nausea and vomiting, pain over the liver area of the body, clay- or grey-colored stool, itching all over, and dark-colored urine, when coupled with a positive test for presence of a hepatitis B virus, a hepatitis B viral antigen, or a positive test for the presence of an antibody specific for a hepatitis B viral antigen.

Certain embodiments provide a method of reducing HBV mRNA expression in an animal comprising administering to the animal a compound or composition described herein. In certain embodiments, the compound or composition comprises a modified oligonucleotide 10 to 30 linked nucleosides in length targeted to HBV. In certain embodiments, reduction of HBV mRNA expression in an animal prevents, ameliorates or treats an HBV-related disease, disorder or condition. In certain embodiments, reduction of HBV mRNA expression in an animal prevents, ameliorates or treats liver disease. In certain embodiments, the HBV mRNA expression is reduced by at least 5%, 10%, 20%, 30%, 35%, 40%, 45%, 50%, 55%, 60%, 65%, 70%, 75%, 80%, 85%, 90%, 95% or 100%.

Certain embodiments provide a method of reducing HBV DNA levels in an animal comprising administering to the animal a compound or composition described herein. In certain embodiments, the compound or composition comprises a modified oligonucleotide 10 to 30 linked nucleosides in length targeted to HBV. In certain embodiments, reduction of HBV DNA levels in an animal prevents, ameliorates or treats an HBV-related disease, disorder or condition. In certain embodiments, the mammal may be human, and the hepatitis B virus may be a human hepatitis B virus. More particularly, the human hepatitis B virus may be any of the human geographical genotypes: A (Northwest Europe, North America, Central America); B (Indonesia, China, Vietnam); C (East Asia, Korea, China, Japan, Polynesia, Vietnam); D (Mediterranean area, Middle East, India); E (Africa); F (Native Americans, Polynesia); G (United States, France); or H (Central America). In certain embodiments, reduction of HBV DNA levels in an animal prevents, ameliorates or treats liver disease. In certain embodiments, the HBV DNA level is reduced by at least 5%, 10%, 20%, 30%, 35%, 40%, 45%, 50%, 55%, 60%, 65%, 70%, 75%, 80%, 85%, 90%, 95% or 100%.

Certain embodiments provide a method of reducing HBV protein levels in an animal comprising administering to the animal a compound or composition described herein. In certain embodiments, the compound or composition comprises a modified oligonucleotide 10 to 30 linked nucleosides in length targeted to HBV. In certain embodiments, reduction of HBV protein levels in an animal prevents, ameliorates or treats an HBV-related disease, disorder or condition. In certain embodiments, reduction of HBV protein levels in an animal prevents, ameliorates or treats liver disease. In certain embodiments, the HBV protein level is reduced by at least 5%, 10%, 20%, 30%, 35%, 40%, 45%, 50%, 55%, 60%, 65%, 70%, 75%, 80%, 85%, 90%, 95% or 100%.

Certain embodiments provide a method of reducing HBV antigen levels in an animal comprising administering to the animal a compound or composition described herein. In certain embodiments, the compound or composition comprises a modified oligonucleotide 10 to 30 linked nucleosides in length targeted to HBV. In certain embodiments, the antigen is HBsAG or HBeAG. In certain embodiments, reduction of HBV antigen levels in an animal prevents, ameliorates or treats an HBV-related disease, disorder or condition. In certain embodiments, reduction of HBV antigen levels in an animal prevents, ameliorates or treats liver disease. In certain embodiments, the HBV antigen levels are reduced by at least 5%, 10%, 20%, 30%, 35%, 40%, 45%, 50%, 55%, 60%, 65%, 70%, 75%, 80%, 85%, 90%, 95% or 100%.

Certain embodiments provide a method of reducing HBV DNA and HBV antigen in a animal infected with a hepatitis B virus, comprising administering to the animal a compound or composition described herein. In certain embodiments, the compound or composition comprises a modified oligonucleotide 10 to 30 linked nucleosides in length targeted to HBV. In certain embodiments, the antigen is HBsAG or HBeAG. In certain embodiments, the amount of HBV antigen may be sufficiently reduced to result in seroconversion, defined as serum HBeAg absence plus serum HBeAb presence if monitoring HBeAg as the determinant for seroconversion, or defined as serum HBsAg absence if monitoring HBsAg as the determinant for seroconversion, as determined by currently available detection limits of commercial ELISA systems.

Certain embodiments provide a method for treating an animal with a HBV related disease, disorder or condition comprising: a) identifying said animal with the HBV related disease, disorder or condition, and b) administering to said animal a therapeutically effective amount of a compound comprising a modified oligonucleotide consisting of 16 to 20 linked nucleosides and having a nucleobase sequence at least 90% complementary to any of SEQ ID NO: 1, 1273, 1274, 1275, 1276, 1277, 1278, 1279, 1280, 1281, 1282, 1283, 1284, 1285, or 1286, as measured over the entirety of said modified oligonucleotide. In certain embodiments, the therapeutically effective amount of the compound administered to the animal treats or reduces the HBV related disease, disorder or condition, or a symptom thereof, in the animal. In certain embodiments, the HBV related disease, disorder or condition is a liver disease. In certain embodiments, the related disease, disorder or condition is chronic HBV infection, jaundice, liver cancer, liver inflammation, liver fibrosis, liver cirrhosis, liver failure, diffuse hepatocellular inflammatory disease, hemophagocytic syndrome, serum hepatitis, HBV viremia, or liver disease-related to transplantation.

In certain embodiments, HBV has the sequence as set forth in GenBank Accession Numbers U95551.1 (incorporated herein as SEQ ID NO: 1) or any variant or fragment thereof. In certain embodiments, HBV has truncated portions of the human sequence as set forth in SEQ ID NOs: 1-14.

In certain embodiments, the animal is a human.

In certain embodiments, the compounds or compositions are designated as a first agent. In certain embodiments, the methods comprise administering a first agent and one or more second agents. In certain embodiments, the methods comprise administering a first agent and one or more second agents. In certain embodiments, the first agent and one or more second agents are co-administered. In certain embodiments the first agent and one or more second agents are co-administered sequentially or concomitantly.

In certain embodiments, the one or more second agents are also a compound or composition described herein. In certain embodiments, the one or more second agents are different from a compound or composition described herein. Examples of one or more second agents include, but are not limited to, an anti-inflammatory agent, chemotherapeutic agent or anti-infection agent.

In other related embodiments, the additional therapeutic agent may be an HBV agent, an HCV agent, a chemotherapeutic agent, an antibiotic, an analgesic, a non-steroidal anti-inflammatory (NSAID) agent, an antifungal agent, an antiparasitic agent, an anti-nausea agent, an anti-diarrheal agent, or an immunosuppressant agent.

In certain embodiments, the one or more second agents are an HBV agent. In certain embodiments the HBV agent can include, but is not limited to, interferon alpha-2b, interferon alpha-2a, and interferon alphacon-1 (pegylated and unpegylated), ribavirin; an HBV RNA replication inhibitor; a second antisense oligomer; an HBV therapeutic vaccine; an HBV prophylactic vaccine; lamivudine (3TC); entecavir (ETV); tenofovir diisoproxil fumarate (TDF); telbivudine (LdT); adefovir; or an HBV antibody therapy (monoclonal or polyclonal).

In certain embodiments, the second agent is an HBV agent. In certain embodiments the HBV agent can include, but is not limited to, interferon alpha-2b, interferon alpha-2a, and interferon alphacon-1 (pegylated and unpegylated), ribavirin; an HBV RNA replication inhibitor; a second antisense oligomer; an HBV therapeutic vaccine; an HBV prophylactic vaccine; lamivudine (3TC); entecavir (ETV); tenofovir diisoproxil fumarate (TDF); telbivudine (LdT); adefovir; or an HBV antibody therapy (monoclonal or polyclonal).

In certain embodiments, the second agent is an HCV agent. In certain embodiments the HBV agent can include, but is not limited to interferon alpha-2b, interferon alpha-2a, and interferon alphacon-1 (pegylated and unpegylated); ribavirin; an HCV RNA replication inhibitor (e.g., ViroPharma's VP50406 series); an HCV antisense agent; an HCV therapeutic vaccine; an HCV protease inhibitor; an HCV helicase inhibitor; or an HCV monoclonal or polyclonal antibody therapy.

In certain embodiments, the second agent is an anti-inflammatory agent (i.e., an inflammation lowering therapy). In certain embodiments the inflammation lowering therapy can include, but is not limited to, a therapeutic lifestyle change, a steroid, a NSAID or a DMARD. The steroid can be a corticosteroid. The NSAID can be an aspirin, acetaminophen, ibuprofen, naproxen, COX inhibitors, indomethacin and the like. The DMARD can be a TNF inhibitor, purine synthesis inhibitor, calcineurin inhibitor, pyrimidine synthesis inhibitor, a sulfasalazine, methotrexate and the like.

In certain embodiments, the second agent is a chemotherapeutic agent (i.e., a cancer treating agent). Chemotherapeutic agents can include, but are not limited to, daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine (CA), 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, taxol, vincristine, vinblastine, etoposide, trimetrexate, teniposide, cisplatin, gemcitabine and diethylstilbestrol (DES).

In certain embodiments, the second agent is an anti-infection agent. Examples of anti-infection agents include, but are not limited to, antibiotics, antifungal drugs and antiviral drugs.

In certain embodiments, administration comprises parenteral administration.

Certain embodiment provides a method for reducing an amount of HBV mRNA, protein and an amount of HBV antigen in a mammal infected with a hepatitis B virus, the method comprising administering a therapeutically effective amount of a pharmaceutical composition as described above to a mammal in need thereof so as to reduce the hepatitis B virus infection and the hepatitis B antigen, compared to the amount of HBV mRNA, protein and an amount of HBV antigen in the mammal before treatment. In some embodiments, the mammal may be human, and the hepatitis B virus may be a human hepatitis B virus. More particularly, the human hepatitis B virus may be any of the human geographical genotypes: A (Northwest Europe, North America, Central America); B (Indonesia, China, Vietnam); C (East Asia, Korea, China, Japan, Polynesia, Vietnam); D (Mediterranean area, Middle East, India); E (Africa); F (Native Americans, Polynesia); G (United States, France); or H (Central America).

In certain embodiments, a method is provided for reducing an amount of HBV mRNA, DNA, protein and/or an amount of HBV antigen in a mammal infected with a hepatitis B virus, the method comprising administering a therapeutically effective amount of a pharmaceutical composition as described above to a mammal in need thereof so as to reduce the hepatitis B virus infection and the hepatitis B antigen, compared to the amount of HBV mRNA, protein and an amount of HBV antigen in the mammal before treatment, wherein the amount of mRNA is reduced at least 70% compared to the amount before administration of the modified antisense oligonucleotide. In certain embodiments, a method is provided for reducing an amount of HBV mRNA, DNA, protein and/or an amount of HBV antigen in a mammal infected with a hepatitis B virus, the method comprising administering a therapeutically effective amount of a pharmaceutical composition as described above to a mammal in need thereof so as to reduce the hepatitis B virus infection and the hepatitis B antigen, compared to the amount of HBV mRNA, protein and an amount of HBV antigen in the mammal before treatment, wherein the amount of mRNA is reduced at least 75% compared to the amount before administration of the modified antisense oligonucleotide. In certain embodiments, a method is provided for reducing an amount of HBV mRNA, DNA, protein and/or an amount of HBV antigen in a mammal infected with a hepatitis B virus, the method comprising administering a therapeutically effective amount of a pharmaceutical composition as described above to a mammal in need thereof so as to reduce the hepatitis B virus infection and the hepatitis B antigen, compared to the amount of HBV mRNA, protein and an amount of HBV antigen in the mammal before treatment, wherein the amount of mRNA is reduced at least 80% compared to the amount before administration of the modified antisense oligonucleotide. In certain embodiments, a method is provided for reducing an amount of HBV mRNA, DNA, protein and/or an amount of HBV antigen in a mammal infected with a hepatitis B virus, the method comprising administering a therapeutically effective amount of a pharmaceutical composition as described above to a mammal in need thereof so as to reduce the hepatitis B virus infection and the hepatitis B antigen, compared to the amount of HBV mRNA, protein and an amount of HBV antigen in the mammal before treatment, wherein the amount of mRNA is reduced at least 85% compared to the amount before administration of the modified antisense oligonucleotide. In certain embodiments, a method is provided for reducing an amount of HBV mRNA, DNA, protein and/or an amount of HBV antigen in a mammal infected with a hepatitis B virus, the method comprising administering a therapeutically effective amount of a pharmaceutical composition as described above to a mammal in need thereof so as to reduce the hepatitis B virus infection and the hepatitis B antigen, compared to the amount of HBV mRNA, protein and an amount of HBV antigen in the mammal before treatment, wherein the amount of mRNA is reduced at least 90% compared to the amount before administration of the modified antisense oligonucleotide. In certain embodiments, a method is provided for reducing an amount of HBV mRNA, DNA, protein and/or an amount of HBV antigen in a mammal infected with a hepatitis B virus, the method comprising administering a therapeutically effective amount of a pharmaceutical composition as described above to a mammal in need thereof so as to reduce the hepatitis B virus infection and the hepatitis B antigen, compared to the amount of HBV mRNA, protein and an amount of HBV antigen in the mammal before treatment, wherein the amount of mRNA is reduced at least 95% compared to the amount before administration of the modified antisense oligonucleotide. In related methods, the HBV antigen may be HBsAg or may be HBeAg, and more particularly, the amount of HBV antigen may be sufficiently reduced to result in seroconversion, defined as serum HBeAg absence plus serum HBeAb presence if monitoring HBeAg as the determinant for seroconversion, or defined as serum HBsAg absence if monitoring HBsAg as the determinant for seroconversion, as determined by currently available detection limits of commercial ELISA systems.

Certain embodiment provides a method for promoting seroconversion of a hepatitis B virus in a mammal infected with HBV, the method comprising administering a therapeutically effective amount of a pharmaceutical composition as described above to a mammal infected with hepatitis B; monitoring for presence of HBeAg plus HBeAb in a serum sample of the mammal, or monitoring for presence of HBsAg in a serum sample of the mammal, such that the absence of HBeAg plus the presence of HBeAb in the serum sample if monitoring HBeAg as the determinant for seroconversion, or the absence of HBsAg in the serum sample if monitoring HBsAg as the determinant for seroconversion, as determined by current detection limits of commercial ELISA systems, is indication of seroconversion in the mammal.

Certain embodiments provide the use of a compound or composition as described herein for preventing, ameliorating or treating liver disease, or symptom thereof, in an animal. In certain embodiments, the compound or composition comprises a modified oligonucleotide 10 to 30 linked nucleosides in length targeted to HBV.

Certain embodiments provide the use of a compound or composition as described herein in the manufacture of a medicament for treating, ameliorating, delaying or preventing an HBV-related disease, disorder or condition in an animal.

Certain embodiments provide the use of a compound or composition as described herein in the manufacture of a medicament for treating, ameliorating, delaying or preventing liver disease in an animal.

Certain embodiments provide a kit for treating, preventing, or ameliorating an HBV-related disease, disorder or condition, or a symptom thereof, as described herein wherein the kit comprises: a) a compound or compositions as described herein; and optionally b) an additional agent or therapy as described herein. The kit can further include instructions or a label for using the kit to treat, prevent, or ameliorate the HBV-related disease, disorder or condition.

In certain embodiments, the compounds or compositions for the described uses or methods comprise a modified oligonucleotide having 10-30 linked nucleosides and a nucleobase sequence comprising at least 10 contiguous nucleobases of any of the nucleobase sequences of SEQ ID NOs: 18-35. In certain embodiments, such oligonucleotides have a gap segment of 10 or more linked deoxynucleosides. In certain embodiments, such gap segment is between two wing segments that independently have 1-5, 1-4, 1-3, 2-5, 2-4 or 2-3 linked modified nucleosides. In certain embodiments, one or more modified nucleosides in the wing segment have a modified sugar. In certain embodiments, the modified sugar is a bicyclic sugar. In certain embodiments, the modified nucleoside is an LNA nucleoside.

### Antisense Compounds

Oligomeric compounds include, but are not limited to, oligonucleotides, oligonucleosides, oligonucleotide analogs, oligonucleotide mimetics, antisense compounds, antisense oligonucleotides, and siRNAs. An oligomeric compound may be “antisense” to a target nucleic acid, meaning that is capable of undergoing hybridization to a target nucleic acid through hydrogen bonding.

In certain embodiments, an antisense compound has a nucleobase sequence that, when written in the 5′ to 3′ direction, comprises the reverse complement of the target segment of a target nucleic acid to which it is targeted. In certain such embodiments, an antisense oligonucleotide has a nucleobase sequence that, when written in the 5′ to 3′ direction, comprises the reverse complement of the target segment of a target nucleic acid to which it is targeted.

In certain embodiments, an antisense compound targeted to a HBV nucleic acid is 10-30 subunits in length. In certain embodiments, an antisense compound targeted to a HBV nucleic acid is 12 to 30. In certain embodiments, an antisense compound targeted to a HBV nucleic acid is 15 to 30 subunits in length. In certain embodiments, an antisense compound targeted to a HBV nucleic acid is 16 to 30 subunits in length. In certain embodiments, an antisense compound targeted to a HBV nucleic acid is 16 subunits in length. In other embodiments, the antisense compound is 8 to 80, 12 to 50, 13 to 30, 13 to 50, 14 to 30, 14 to 50, 15 to 30, 15 to 50, 16 to 30, 16 to 50, 17 to 30, 17 to 50, 18 to 22, 18 to 24, 18 to 30, 18 to 50, 19 to 22, 19 to 30, 19 to 50, or 20 to 30 linked subunits. In certain such embodiments, the antisense compounds are 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked subunits in length, or a range defined by any two of the above values. In some embodiments the antisense compound is an antisense oligonucleotide, and the linked subunits are nucleotides.

In certain embodiments antisense oligonucleotides targeted to a HBV nucleic acid may be shortened or truncated. For example, a single subunit may be deleted from the 5′ end (5′ truncation), or alternatively from the 3′ end (3′ truncation). A shortened or truncated antisense compound targeted to a HBV nucleic acid may have two subunits deleted from the 5′ end, or alternatively may have two subunits deleted from the 3′ end, of the antisense compound. Alternatively, the deleted nucleosides may be dispersed throughout the antisense compound, for example, in an antisense compound having one nucleoside deleted from the 5′ end and one nucleoside deleted from the 3′ end.

When a single additional subunit is present in a lengthened antisense compound, the additional subunit may be located at the 5′ or 3′ end of the antisense compound. When two or more additional subunits are present, the added subunits may be adjacent to each other, for example, in an antisense compound having two subunits added to the 5′ end (5′ addition), or alternatively to the 3′ end (3′ addition), of the antisense compound. Alternatively, the added subunits may be dispersed throughout the antisense compound, for example, in an antisense compound having one subunit added to the 5′ end and one subunit added to the 3′ end.

It is possible to increase or decrease the length of an antisense compound, such as an antisense oligonucleotide, and/or introduce mismatch bases without eliminating activity. For example, in Woolf et al. (Proc. Natl. Acad. Sci. USA 89:7305-7309, 1992), a series of antisense oligonucleotides 13-25 nucleobases in length were tested for their ability to induce cleavage of a target RNA in an oocyte injection model. Antisense oligonucleotides 25 nucleobases in length with 8 or 11 mismatch bases near the ends of the antisense oligonucleotides were able to direct specific cleavage of the target mRNA, albeit to a lesser extent than the antisense oligonucleotides that contained no mismatches. Similarly, target specific cleavage was achieved using 13 nucleobase antisense oligonucleotides, including those with 1 or 3 mismatches.

Gautschi et al. (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo.

Maher and Dolnick (Nuc. Acid. Res. 16:3341-3358, 1988) tested a series of tandem 14 nucleobase antisense oligonucleotides, and a 28 and 42 nucleobase antisense oligonucleotides comprised of the sequence of two or three of the tandem antisense oligonucleotides, respectively, for their ability to arrest translation of human DHFR in a rabbit reticulocyte assay. Each of the three 14 nucleobase antisense oligonucleotides alone was able to inhibit translation, albeit at a more modest level than the 28 or 42 nucleobase antisense oligonucleotides.

In certain embodiments, such compounds include compounds comprising the nucleobase sequence of any one of SEQ ID NOs: 18-35.

### Antisense Compound Motifs

In certain embodiments, antisense compounds targeted to a HBV nucleic acid have chemically modified subunits arranged in patterns, or motifs, to confer to the antisense compounds properties such as enhanced inhibitory activity, increased binding affinity for a target nucleic acid, or resistance to degradation by in vivo nucleases.

Chimeric antisense compounds typically contain at least one region modified so as to confer increased resistance to nuclease degradation, increased cellular uptake, increased binding affinity for the target nucleic acid, and/or increased inhibitory activity. A second region of a chimeric antisense compound may optionally serve as a substrate for the cellular endonuclease RNase H, which cleaves the RNA strand of an RNA:DNA duplex.

Antisense compounds having a gapmer motif are considered chimeric antisense compounds. In a gapmer an internal region having a plurality of nucleotides that supports RNaseH cleavage is positioned between external regions having a plurality of nucleotides that are chemically distinct from the nucleosides of the internal region. In the case of an antisense oligonucleotide having a gapmer motif, the gap segment generally serves as the substrate for endonuclease cleavage, while the wing segments comprise modified nucleosides. In certain embodiments, the regions of a gapmer are differentiated by the types of sugar moieties comprising each distinct region. The types of sugar moieties that are used to differentiate the regions of a gapmer may in some embodiments include β-D-ribonucleosides, β-D-deoxyribonucleosides, and bicyclic sugar modified nucleosides. In certain embodiments, wings may include several modified sugar moieties, including, for example LNA. In certain embodiments, wings may include several modified and unmodified sugar moieties. In certain embodiments, wings may include various combinations of LNA nucleosides and 2′-deoxynucleosides.

Each distinct region may comprise uniform sugar moieties, variant, or alternating sugar moieties. The wing-gap-wing motif is frequently described as “X-Y-Z”, where “X” represents the length of the 5′-wing, “Y” represents the length of the gap, and “Z” represents the length of the 3′-wing. “X” and “Z” may comprise uniform, variant, or alternating sugar moieties. In certain embodiments, “X” and “Y” may include one or more 2′-deoxynucleosides. “Y” may comprise 2′-deoxynucleosides. As used herein, a gapmer described as “X-Y-Z” has a configuration such that the gap is positioned immediately adjacent to each of the 5′-wing and the 3′ wing. Thus, no intervening nucleotides exist between the 5′-wing and gap, or the gap and the 3′-wing. Any of the antisense compounds described herein can have a gapmer motif. In certain embodiments, “X” and “Z” are the same; in other embodiments they are different. In certain embodiments, “Y” is between 8 and 15 nucleosides. X, Y, or Z can be any of 1, 2, 3, 4, 5, 6, 7, 8, 9, 10, 11, 12, 13, 14, 15, 16, 17, 18, 19, 20, 25, 30 or more nucleosides.

In certain embodiments, gapmers provided herein include, for example, 16-mers. In certain embodiments, the 16mer has a gap of 10 linked nucleosides. In certain embodiments the gapmer has a motif of or 3-10-3.

In certain embodiments, gapmers provided herein include, for example, 14-mers. In certain embodiments, the 14mers have a gap of 10 linked nucleosides. In certain embodiments, the gapmers have a motif of 2-10-2.

In certain embodiments, the antisense compound targeted to a HBV nucleic acid has a 3-10-3 gapmer motif.

In certain embodiments, the antisense compound targeted to a HBV nucleic acid has a 2-10-2 gapmer motif.

### Target Nucleic Acids, Target Regions and Nucleotide Sequences

Nucleotide sequences that encode HBV include, without limitation, the following: GENBANK Accession U95551.1 (incorporated herein as SEQ ID NO: 1).

It is understood that the sequence set forth in each SEQ ID NO in the Examples contained herein is independent of any modification to a sugar moiety, an internucleoside linkage, or a nucleobase. As such, antisense compounds defined by a SEQ ID NO may comprise, independently, one or more modifications to a sugar moiety, an internucleoside linkage, or a nucleobase. Antisense compounds described by Isis Number (Isis No) indicate a combination of nucleobase sequence and motif.

In certain embodiments, a target region is a structurally defined region of the target nucleic acid. For example, a target region may encompass a 3′ UTR, a 5′ UTR, an exon, an intron, an exon/intron junction, a coding region, a translation initiation region, translation termination region, or other defined nucleic acid region. The structurally defined regions for HBV can be obtained by accession number from sequence databases such as NCBI and such information is incorporated herein by reference. In certain embodiments, a target region may encompass the sequence from a 5′ target site of one target segment within the target region to a 3′ target site of another target segment within the same target region.

Targeting includes determination of at least one target segment to which an antisense compound hybridizes, such that a desired effect occurs. In certain embodiments, the desired effect is a reduction in mRNA target nucleic acid levels. In certain embodiments, the desired effect is reduction of levels of protein encoded by the target nucleic acid or a phenotypic change associated with the target nucleic acid.

A target region may contain one or more target segments. Multiple target segments within a target region may be overlapping. Alternatively, they may be non-overlapping. In certain embodiments, target segments within a target region are separated by no more than about 300 nucleotides. In certain embodiments, target segments within a target region are separated by a number of nucleotides that is, is about, is no more than, is no more than about, 250, 200, 150, 100, 90, 80, 70, 60, 50, 40, 30, 20, or 10 nucleotides on the target nucleic acid, or is a range defined by any two of the preceeding values. In certain embodiments, target segments within a target region are separated by no more than, or no more than about, 5 nucleotides on the target nucleic acid. In certain embodiments, target segments are contiguous. Contemplated are target regions defined by a range having a starting nucleic acid that is any of the 5′ target sites or 3′ target sites listed herein.

Suitable target segments may be found within a 5′ UTR, a coding region, a 3′ UTR, an intron, an exon, or an exon/intron junction. Target segments containing a start codon or a stop codon are also suitable target segments. A suitable target segment may specifically exclude a certain structurally defined region such as the start codon or stop codon.

The determination of suitable target segments may include a comparison of the sequence of a target nucleic acid to other sequences throughout the genome. For example, the BLAST algorithm may be used to identify regions of similarity amongst different nucleic acids. This comparison can prevent the selection of antisense compound sequences that may hybridize in a non-specific manner to sequences other than a selected target nucleic acid (i.e., non-target or off-target sequences).

There may be variation in activity (e.g., as defined by percent reduction of target nucleic acid levels) of the antisense compounds within an active target region. In certain embodiments, reductions in HBV mRNA levels are indicative of inhibition of HBV expression. Reductions in levels of a HBV protein are also indicative of inhibition of target mRNA expression. Further, phenotypic changes are indicative of inhibition of HBV expression. In certain embodiments, reduced fatigue, reduced flu-like symptoms, increase in appetite, reduced nausea, reduced joint pain, reduced jaundice, reduced pain in the abdomen, reduced weakness, reduced weight loss, reduction in breast enlargement in men, reduced rash on the palms, reduced difficulty with blood clotting, reduced cirrhosis, reduced spider-like blood vessels on the skin, increased Vitamins A and D absorption, reduced tumor growth, reduced tumor volume, reduced headache, reduced fever, reduced diarrhea, reduced pain over the liver area of the body, reduced clay- or grey-colored stool, reduced itching, reduced dark-colored urine, and reduced nausea and vomiting can be indicative of inhibition of HBV expression, In certain embodiments, amelioration of symptoms associated with HBV-related conditions, disease, and disorders can be indicative of inhibition of HBV expression. In certain embodiments, reduction of cirrhosis is indicative of inhibition of HBV expression. In certain embodiments, reduction of liver cancer markers can be indicative of inhibition of HBV expression.

### Hybridization

In some embodiments, hybridization occurs between an antisense compound disclosed herein and a HBV nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Stringent conditions are sequence-dependent and are determined by the nature and composition of the nucleic acid molecules to be hybridized.

Methods of determining whether a sequence is specifically hybridizable to a target nucleic acid are well known in the art. In certain embodiments, the antisense compounds provided herein are specifically hybridizable with a HBV nucleic acid.

### Complementarity

An antisense compound and a target nucleic acid are complementary to each other when a sufficient number of nucleobases of the antisense compound can hydrogen bond with the corresponding nucleobases of the target nucleic acid, such that a desired effect will occur (e.g., antisense inhibition of a target nucleic acid, such as a HBV nucleic acid).

Non-complementary nucleobases between an antisense compound and a HBV nucleic acid may be tolerated provided that the antisense compound remains able to specifically hybridize to a target nucleic acid. Moreover, an antisense compound may hybridize over one or more segments of a HBV nucleic acid such that intervening or adjacent segments are not involved in the hybridization event (e.g., a loop structure, mismatch or hairpin structure).

In certain embodiments, the antisense compounds provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to a HBV nucleic acid, a target region, target segment, or specified portion thereof. Percent complementarity of an antisense compound with a target nucleic acid can be determined using routine methods.

For example, an antisense compound in which 18 of 20 nucleobases of the antisense compound are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an antisense compound which is 18 nucleobases in length having four noncomplementary nucleobases which are flanked by two regions of complete complementarity with the target nucleic acid would have 77.8% overall complementarity with the target nucleic acid and would thus fall within the scope of the present invention. Percent complementarity of an antisense compound with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, the antisense compounds provided herein, or specified portions thereof, are fully complementary (i.e. 100% complementary) to a target nucleic acid, or specified portion thereof. For example, an antisense compound may be fully complementary to a HBV nucleic acid, or a target region, or a target segment or target sequence thereof. As used herein, “fully complementary” means each nucleobase of an antisense compound is capable of precise base pairing with the corresponding nucleobases of a target nucleic acid. For example, a 20 nucleobase antisense compound is fully complementary to a target sequence that is 400 nucleobases long, so long as there is a corresponding 20 nucleobase portion of the target nucleic acid that is fully complementary to the antisense compound. Fully complementary can also be used in reference to a specified portion of the first and/or the second nucleic acid. For example, a 20 nucleobase portion of a 30 nucleobase antisense compound can be “fully complementary” to a target sequence that is 400 nucleobases long. The 20 nucleobase portion of the 30 nucleobase oligonucleotide is fully complementary to the target sequence if the target sequence has a corresponding 20 nucleobase portion wherein each nucleobase is complementary to the 20 nucleobase portion of the antisense compound. At the same time, the entire 30 nucleobase antisense compound may or may not be fully complementary to the target sequence, depending on whether the remaining 10 nucleobases of the antisense compound are also complementary to the target sequence.

The location of a non-complementary nucleobase may be at the 5′ end or 3′ end of the antisense compound. Alternatively, the non-complementary nucleobase or nucleobases may be at an internal position of the antisense compound. When two or more non-complementary nucleobases are present, they may be contiguous (i.e. linked) or non-contiguous. In one embodiment, a non-complementary nucleobase is located in the wing segment of a gapmer antisense oligonucleotide.

In certain embodiments, antisense compounds that are, or are up to 11, 12, 13, 14, 15, 16, 17, 18, 19, or 20 nucleobases in length comprise no more than 4, no more than 3, no more than 2, or no more than 1 non-complementary nucleobase(s) relative to a target nucleic acid, such as a HBV nucleic acid, or specified portion thereof.

In certain embodiments, antisense compounds that are, or are up to 11, 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, such as a HBV nucleic acid, or specified portion thereof.

The antisense compounds provided also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of an antisense compound. In certain embodiments, the antisense compounds, are complementary to at least an 8 nucleobase portion of a target segment. In certain embodiments, the antisense compounds are complementary to at least a 9 nucleobase portion of a target segment. In certain embodiments, the antisense compounds are complementary to at least a 10 nucleobase portion of a target segment. In certain embodiments, the antisense compounds are complementary to at least an 11 nucleobase portion of a target segment. In certain embodiments, the antisense compounds are complementary to at least a 12 nucleobase portion of a target segment. In certain embodiments, the antisense compounds are complementary to at least a 13 nucleobase portion of a target segment. In certain embodiments, the antisense compounds are complementary to at least a 14 nucleobase portion of a target segment. In certain embodiments, the antisense compounds are complementary to at least a 15 nucleobase portion of a target segment. In certain embodiments, the antisense compounds are complementary to at least a 16 nucleobase portion of a target segment. Also contemplated are antisense compounds that are complementary to at least a 9, 10, 11, 12, 13, 14, 15, 16, 17, 18, 19, 20, or more nucleobase portion of a target segment, or a range defined by any two of these values.

### Identity

The antisense compounds provided herein may also have a defined percent identity to a particular nucleotide sequence, SEQ ID NO, or compound represented by a specific Isis number, or portion thereof. As used herein, an antisense compound is identical to the sequence disclosed herein if it has the same nucleobase pairing ability. For example, a RNA which contains uracil in place of thymidine in a disclosed DNA sequence would be considered identical to the DNA sequence since both uracil and thymidine pair with adenine. Shortened and lengthened versions of the antisense compounds described herein as well as compounds having non-identical bases relative to the antisense compounds provided herein also are contemplated. The non-identical bases may be adjacent to each other or dispersed throughout the antisense compound. Percent identity of an antisense compound is calculated according to the number of bases that have identical base pairing relative to the sequence to which it is being compared.

In certain embodiments, the antisense compounds, or portions thereof, are at least 70%, 75%, 80%, 85%, 90%, 95%, 96%, 97%, 98%, 99% or 100% identical to one or more of the antisense compounds or SEQ ID NOs, or a portion thereof, disclosed herein.

In certain embodiments, a portion of the antisense compound is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

In certain embodiments, a portion of the antisense oligonucleotide is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

### Modifications

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to antisense compounds encompass substitutions or changes to internucleoside linkages, sugar moieties, or nucleobases. Modified antisense compounds are often preferred over native forms because of desirable properties such as, for example, enhanced cellular uptake, enhanced affinity for nucleic acid target, increased stability in the presence of nucleases, or increased inhibitory activity.

Chemically modified nucleosides may also be employed to increase the binding affinity of a shortened or truncated antisense oligonucleotide for its target nucleic acid. Consequently, comparable results can often be obtained with shorter antisense compounds that have such chemically modified nucleosides.

### Modified Internucleoside Linkages

The naturally occurring internucleoside linkage of RNA and DNA is a 3′ to 5′ phosphodiester linkage. Antisense compounds having one or more modified, i.e. non-naturally occurring, internucleoside linkages are often selected over antisense compounds having naturally occurring internucleoside linkages because of desirable properties such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, antisense compounds targeted to a HBV nucleic acid comprise one or more modified internucleoside linkages. In certain embodiments, the modified internucleoside linkages are phosphorothioate linkages. In certain embodiments, each internucleoside linkage of an antisense compound is a phosphorothioate internucleoside linkage.

### Modified Sugar Moieties

Oligomeric compounds provided herein may comprise one or more monomers, including a nucleoside or nucleotide, having a modified sugar moiety. For example, the furanosyl sugar ring of a nucleoside or nucleotide can be modified in a number of ways including, but not limited to, addition of a substituent group and bridging of two non-geminal ring atoms to form a Locked Nucleic Acid (LNA).

In certain embodiments, oligomeric compounds comprise one or more monomers having a bicyclic sugar. In certain embodiments, the monomer is an LNA. In certain such embodiments, LNAs include, but are not limited to, (A) α-L-Methyleneoxy (4′-CH₂—O-2′) LNA, (B) β-D-Methyleneoxy (4′-CH₂—O-2′) LNA, (C) Ethyleneoxy (4′-(CH₂)₂—O-2′) LNA, (D) Aminooxy (4′-CH₂—O—N(R)-2′) LNA and (E) Oxyamino (4′-CH₂—N(R)—O-2′) LNA, as depicted below.

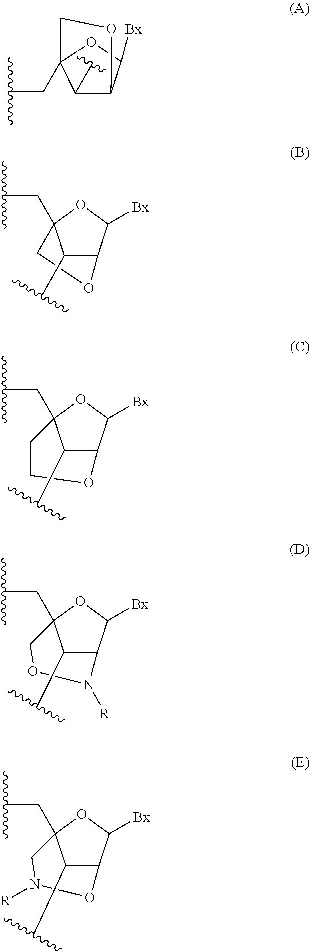

In certain embodiments, LNA compounds include, but are not limited to, compounds having at least one bridge between the 4′ and the 2′ position of the sugar wherein each of the bridges independently comprises 1 or from 2 to 4 linked groups independently selected from —[C(R₁)(R₂)]ₙ—, —C(R₁)═C(R₂)—, —C(R₁)═N—, —C(═NR₁)—, —C(═O)—, —C(═S)—, —O—, —Si(R₁)₂—, —S(═O)ₓ— and —N(R₁)—;

wherein:

x is 0, 1, or 2;

n is 1, 2, 3, or 4;

each R₁ and R₂ is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, a heterocycle radical, a substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and

each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

In one embodiment, each of the bridges of the LNA compounds is, independently, —[C(R₁)(R₂)]ₙ—, —[C(R₁)(R₂)]ₙ—O—, —C(R₁R₂)—N(R₁)—O— or —C(R₁R₂)—O—N(R₁)—. In another embodiment, each of said bridges is, independently, 4′-CH₂-2′,4′-(CH₂)₂-2′,4′-(CH₂)₃-2′,4′-CH₂—O-2′,4′-(CH₂)₂—O-2′, 4′-CH₂—O—N(R₁)-2′ and 4′-CH₂—N(R₁)—O-2′- wherein each R₁ is, independently, H, a protecting group or C₁-C₁₂ alkyl.

Certain LNA's have been prepared and disclosed in the patent literature as well as in scientific literature (see for example: issued U.S. Pat. Nos. 7,053,207; 6,268,490; 6,770,748; 6,794,499; 7,034,133; 6,525,191; 7,696,345; 7,569,575; 7,314,923; 7,217,805; and 7,084,125, hereby incorporated by reference herein in their entirety.

Also provided herein are LNAs in which the 2′-hydroxyl group is connected, to the 4′ carbon atom of the ribosyl sugar ring, thereby forming a methyleneoxy (4′-CH₂—O-2′) bridge to form the bicyclic sugar moiety (reviewed in Elayadi et al., Curr. Opinion Invens. Drugs, 2001, 2, 558-561; Braasch et al., Chem. Biol., 2001, 8 1-7; and Orum et al., Curr. Opinion Mol. Ther., 2001, 3, 239-243; see also U.S. Pat. No. 6,670,461). Furthermore, the bridge can also be a methylene (—CH₂—) group connecting the 2′ oxygen atom to the 4′ carbon atom of the sugar ring, for which the term methyleneoxy (4′-CH₂—O-2′) LNA is used. In the case of the bicylic sugar moiety having an ethylene bridging group in this position, the term ethyleneoxy (4′-CH₂CH₂—O-2′) LNA is used (Singh et al., Chem. Commun., 1998, 4, 455-456: Morita et al., Bioorganic Medicinal Chemistry, 2003, 11, 2211-2226). Methyleneoxy (4′-CH₂—O-2′) LNA and other bicyclic sugar analogs display very high duplex thermal stabilities with complementary DNA and RNA (Tm=+3 to +10° C.), stability towards 3′-exonucleolytic degradation and good solubility properties. Potent and nontoxic antisense oligonucleotides comprising LNAs have been described (Wahlestedt et al., Proc. Natl. Acad. Sci. U.S.A., 2000, 97, 5633-5638).

An isomer of methyleneoxy (4′-CH₂—O-2′) LNA that has also been discussed is α-L-methyleneoxy (4′-CH₂—O-2′) LNA which has been shown to have superior stability against a 3′-exonuclease. The α-L-methyleneoxy (4′-CH₂—O-2′) LNA's were incorporated into antisense gapmers and chimeras that showed potent antisense activity (Frieden et al., Nucleic Acids Research, 2003, 21, 6365-6372).

The synthesis and preparation of adenine, cytosine, guanine, 5-methyl-cytosine, thymine and uracil LNAs having a methyleneoxy (4′-CH₂—O-2′) bridge, along with their oligomerization, and nucleic acid recognition properties have been described (Koshkin et al., Tetrahedron, 1998, 54, 3607-3630). LNAs and preparation thereof are also described in WO 98/39352 and WO 99/14226, incorporated by reference herein.

Analogs of various LNA nucleosides that have 4′ to 2′ bridging groups such as 4′-CH₂—O-2′ (methyleneoxy) and 4′-CH₂—S-2′ (methylene-thio), have also been prepared (Kumar et al., Bioorg. Med. Chem. Lett., 1998, 8, 2219-2222). Preparation of locked nucleoside analogs comprising oligodeoxyribo-nucleotide duplexes as substrates for nucleic acid polymerases has also been described (Wengel et al., WO 99/14226). Furthermore, synthesis of 2′-amino-LNA, a novel comformationally restricted high-affinity oligonucleotide analog has been described in the art (Singh et al., J. Org. Chem., 1998, 63, 10035-10039). In addition, 2′-amino- and 2′-methylamino-LNA's have been prepared and the thermal stability of their duplexes with complementary RNA and DNA strands has been previously reported.

In one embodiment, the antisense oligomer may comprise at least two LNA monomers, more particularly the antisense oligomer may comprise 2, 3, 4, 5, 6, 7, 8, 9 or 10 LNA monomers. As described below, the contiguous nucleotide sequence may consist of LNA and DNA units (including linkage groups, such as phosphorothioate linkages), or may consist of LNA and other nucleoside and nucleotide analogues. In some embodiments, the contiguous nucleotide sequence may comprise 6, 7, 8, 9, 10, 11, 12, 13, 14, 15, or 16 DNA nucleosides, the remainder of the monomers in the antisense oligomer comprising nucleoside analogues, such as LNAs.

As used herein, “bicyclic nucleoside” refers to a nucleoside comprising a bridge connecting two carbon atoms of the sugar ring, thereby forming a bicyclic sugar moiety. In certain embodiments, the bridge connects the 2′ carbon and another carbon of the sugar ring.

As used herein, “4′-2′ bicyclic nucleoside” or “4′ to 2′ bicyclic nucleoside” refers to a bicyclic nucleoside comprising a furanose ring comprising a bridge connecting the 2′ carbon atom and the 4′ carbon atom of the sugar ring.

One carbocyclic bicyclic nucleoside having a 4′-(CH₂)₃-2′ bridge and the alkenyl analog, bridge 4′-CH═CH—CH₂-2′, have been described (see, e.g., Freier et al., Nucleic Acids Research, 1997, 25(22), 4429-4443 and Albaek et al., J. Org. Chem., 2006, 71, 7731-7740). The synthesis and preparation of carbocyclic bicyclic nucleosides along with their oligomerization and biochemical studies have also been described (see, e.g., Srivastava et al., J. Am. Chem. Soc. 2007, 129(26), 8362-8379).

Methods for the preparations of modified sugars are well known to those skilled in the art.

### Compositions and Methods for Formulating Pharmaceutical Compositions

Antisense oligonucleotides may be admixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

An antisense compound targeted to a HBV nucleic acid can be utilized in pharmaceutical compositions by combining the antisense compound with a suitable pharmaceutically acceptable diluent or carrier. A pharmaceutically acceptable diluent includes phosphate-buffered saline (PBS). PBS is a diluent suitable for use in compositions to be delivered parenterally. Accordingly, in one embodiment, employed in the methods described herein is a pharmaceutical composition comprising an antisense compound targeted to a HBV nucleic acid and a pharmaceutically acceptable diluent. In certain embodiments, the pharmaceutically acceptable diluent is PBS. In certain embodiments, the antisense compound is an antisense oligonucleotide.

Pharmaceutical compositions comprising antisense compounds encompass any pharmaceutically acceptable salts, esters, or salts of such esters, or any other oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of an antisense compound which are cleaved by endogenous nucleases within the body, to form the active antisense compound.

### Conjugated Antisense Compounds

Antisense compounds may be covalently linked to one or more moieties or conjugates which enhance the activity, cellular distribution or cellular uptake of the resulting antisense oligonucleotides. Typical conjugate groups include cholesterol moieties and lipid moieties. Additional conjugate groups include carbohydrates, phospholipids, biotin, phenazine, folate, phenanthridine, anthraquinone, acridine, fluoresceins, rhodamines, coumarins, and dyes.

Antisense compounds can also be modified to have one or more stabilizing groups that are generally attached to one or both termini of antisense compounds to enhance properties such as, for example, nuclease stability. Included in stabilizing groups are cap structures. These terminal modifications protect the antisense compound having terminal nucleic acid from exonuclease degradation, and can help in delivery and/or localization within a cell. The cap can be present at the 5′-terminus (5′-cap), or at the 3′-terminus (3′-cap), or can be present on both termini. Cap structures are well known in the art and include, for example, inverted deoxy abasic caps. Further 3′ and 5′-stabilizing groups that can be used to cap one or both ends of an antisense compound to impart nuclease stability include those disclosed in WO 03/004602 published on Jan. 16, 2003.

### Cell Culture and Antisense Compounds Treatment

The effects of antisense compounds on the level, activity or expression of HBV nucleic acids can be tested in vitro in a variety of cell types. Cell types used for such analyses are available from commerical vendors (e.g. American Type Culture Collection, Manassus, Va.; Zen-Bio, Inc., Research Triangle Park, N.C.; Clonetics Corporation, Walkersville, Md.) and are cultured according to the vendor's instructions using commercially available reagents (e.g. Invitrogen Life Technologies, Carlsbad, Calif.). Illustrative cell types include, but are not limited to, HuVEC cells, b.END cells, HepG2 cells, Hep3B cells, and primary hepatocytes.

### In Vitro Testing of Antisense Oligonucleotides

Described herein are methods for treatment of cells with antisense oligonucleotides, which can be modified appropriately for treatment with other antisense compounds.

Cells may be treated with antisense oligonucleotides when the cells reach approximately 60-80% confluency in culture.

One reagent commonly used to introduce antisense oligonucleotides into cultured cells includes the cationic lipid transfection reagent LIPOFECTIN (Invitrogen, Carlsbad, Calif.). Antisense oligonucleotides may be mixed with LIPOFECTIN in OPTI-MEM 1 (Invitrogen, Carlsbad, Calif.) to achieve the desired final concentration of antisense oligonucleotide and a LIPOFECTIN concentration that may range from 2 to 12 ug/mL per 100 nM antisense oligonucleotide.

Another reagent used to introduce antisense oligonucleotides into cultured cells includes LIPOFECTAMINE (Invitrogen, Carlsbad, Calif.). Antisense oligonucleotide is mixed with LIPOFECTAMINE in OPTI-MEM 1 reduced serum medium (Invitrogen, Carlsbad, Calif.) to achieve the desired concentration of antisense oligonucleotide and a LIPOFECTAMINE concentration that may range from 2 to 12 ug/mL per 100 nM antisense oligonucleotide.

Another technique used to introduce antisense oligonucleotides into cultured cells includes electroporation.

Cells are treated with antisense oligonucleotides by routine methods. Cells may be harvested 16-24 hours after antisense oligonucleotide treatment, at which time RNA or protein levels of target nucleic acids are measured by methods known in the art and described herein. In general, when treatments are performed in multiple replicates, the data are presented as the average of the replicate treatments.

The concentration of antisense oligonucleotide used varies from cell line to cell line. Methods to determine the optimal antisense oligonucleotide concentration for a particular cell line are well known in the art. Antisense oligonucleotides are typically used at concentrations ranging from 1 nM to 300 nM when transfected with LIPOFECTAMINE. Antisense oligonucleotides are used at higher concentrations ranging from 625 to 20,000 nM when transfected using electroporation.

### RNA Isolation

RNA analysis can be performed on total cellular RNA or poly(A)+ mRNA. Methods of RNA isolation are well known in the art. RNA is prepared using methods well known in the art, for example, using the TRIZOL Reagent (Invitrogen, Carlsbad, Calif.) according to the manufacturer's recommended protocols.

### Analysis of Inhibition of Target Levels or Expression

Inhibition of levels or expression of a HBV nucleic acid can be assayed in a variety of ways known in the art. For example, target nucleic acid levels can be quantitated by, e.g., Northern blot analysis, competitive polymerase chain reaction (PCR), or quantitaive real-time PCR. RNA analysis can be performed on total cellular RNA or poly(A)+ mRNA. Methods of RNA isolation are well known in the art. Northern blot analysis is also routine in the art. Quantitative real-time PCR can be conveniently accomplished using the commercially available ABI PRISM 7600, 7700, or 7900 Sequence Detection System, available from PE-Applied Biosystems, Foster City, Calif. and used according to manufacturer's instructions.

### Quantitative Real-Time PCR Analysis of Target RNA Levels

Quantitation of target RNA levels may be accomplished by quantitative real-time PCR using the ABI PRISM 7600, 7700, or 7900 Sequence Detection System (PE-Applied Biosystems, Foster City, Calif.) according to manufacturer's instructions. Methods of quantitative real-time PCR are well known in the art.

Prior to real-time PCR, the isolated RNA is subjected to a reverse transcriptase (RT) reaction, which produces complementary DNA (cDNA) that is then used as the substrate for the real-time PCR amplification. The RT and real-time PCR reactions are performed sequentially in the same sample well. RT and real-time PCR reagents may be obtained from Invitrogen (Carlsbad, Calif.). RT real-time-PCR reactions are carried out by methods well known to those skilled in the art.

Gene (or RNA) target quantities obtained by real time PCR are normalized using either the expression level of a gene whose expression is constant, such as cyclophilin A, or by quantifying total RNA using RIBOGREEN (Invitrogen, Inc. Carlsbad, Calif.). Cyclophilin A expression is quantified by real time PCR, by being run simultaneously with the target, multiplexing, or separately. Total RNA is quantified using RIBOGREEN RNA quantification reagent (Invetrogen, Inc. Eugene, Oreg.). Methods of RNA quantification by RIBOGREEN are taught in Jones, L. J., et al, (Analytical Biochemistry, 1998, 265, 368-374). A CYTOFLUOR 4000 instrument (PE Applied Biosystems) is used to measure RIBOGREEN fluorescence.

Probes and primers are designed to hybridize to a HBV nucleic acid. Methods for designing real-time PCR probes and primers are well known in the art, and may include the use of software such as PRIMER EXPRESS Software (Applied Biosystems, Foster City, Calif.).

### Quantitative Real-Time PCR Analysis of Target DNA Levels

Quantitation of target DNA levels may be accomplished by quantitative real-time PCR using the ABI PRISM 7600, 7700, or 7900 Sequence Detection System (PE-Applied Biosystems, Foster City, Calif.) according to manufacturer's instructions. Methods of quantitative real-time PCR are well known in the art.

Gene (or DNA) target quantities obtained by real time PCR are normalized using either the expression level of a gene whose expression is constant, such as cyclophilin A, or by quantifying total DNA using RIBOGREEN (Invitrogen, Inc. Carlsbad, Calif.). Cyclophilin A expression is quantified by real time PCR, by being run simultaneously with the target, multiplexing, or separately. Total DNA is quantified using RIBOGREEN RNA quantification reagent (Invetrogen, Inc. Eugene, Oreg.). Methods of DNA quantification by RIBOGREEN are taught in Jones, L. J., et al, (Analytical Biochemistry, 1998, 265, 368-374). A CYTOFLUOR 4000 instrument (PE Applied Biosystems) is used to measure RIBOGREEN fluorescence.

Probes and primers are designed to hybridize to a HBV nucleic acid. Methods for designing real-time PCR probes and primers are well known in the art, and may include the use of software such as PRIMER EXPRESS Software (Applied Biosystems, Foster City, Calif.).

### Analysis of Protein Levels

Antisense inhibition of HBV nucleic acids can be assessed by measuring HBV protein levels. Protein levels of HBV can be evaluated or quantitated in a variety of ways well known in the art, such as immunoprecipitation, Western blot analysis (immunoblotting), enzyme-linked immunosorbent assay (ELISA), quantitative protein assays, protein activity assays (for example, caspase activity assays), immunohistochemistry, immunocytochemistry or fluorescence-activated cell sorting (FACS). Antibodies directed to a target can be identified and obtained from a variety of sources, such as the MSRS catalog of antibodies (Aerie Corporation, Birmingham, Mich.), or can be prepared via conventional monoclonal or polyclonal antibody generation methods well known in the art.

### In Vivo Testing of Antisense Compounds

Antisense compounds, for example, antisense oligonucleotides, are tested in animals to assess their ability to inhibit expression of HBV and produce phenotypic changes. Testing may be performed in normal animals, or in experimental disease models. For administration to animals, antisense oligonucleotides are formulated in a pharmaceutically acceptable diluent, such as phosphate-buffered saline. Administration includes parenteral routes of administration, such as intraperitoneal, intravenous, subcutaneous, intrathecal, and intracerebroventricular. Calculation of antisense oligonucleotide dosage and dosing frequency is within the abilities of those skilled in the art, and depends upon factors such as route of administration and animal body weight. Following a period of treatment with antisense oligonucleotides, RNA is isolated from liver tissue and changes in HBV nucleic acid expression are measured. Changes in HBV DNA levels are also measured. Changes in HBV protein levels are also measured.

### Certain Indications

In certain embodiments, provided herein are methods, compounds, and compositions of treating an individual comprising administering one or more pharmaceutical compositions provided herein. In certain embodiments, the individual has an HBV-related condition. In certain embodiments, inflammation, fibrosis, cirrhosis, liver cancer, serum hepatitis, jaundice, liver cancer, liver inflammation, liver fibrosis, liver cirrhosis, liver failure, diffuse hepatocellular inflammatory disease, hemophagocytic syndrome, serum hepatitis, and HBV viremia. In certain embodiments, the HBV-related condition may have which may include any or all of the following: flu-like illness, weakness, aches, headache, fever, loss of appetite, diarrhea, jaundice, nausea and vomiting, pain over the liver area of the body, clay- or grey-colored stool, itching all over, and dark-colored urine, when coupled with a positive test for presence of a hepatitis B virus, a hepatitis B viral antigen, or a positive test for the presence of an antibody specific for a hepatitis B viral antigen. In certain embodiments, the individual is at risk for an HBV-related condition. This includes individuals having one or more risk factors for developing an HBV-related condition, including sexual exposure to an individual infected with Hepatitis B virus, living in the same house as an individual with a lifelong hepatitis B virus infection, exposure to human blood infected with the hepatitis B virus, injection of illicit drugs, being a person who has hemophilia, and visiting an area where hepatitis B is common. In certain embodiments, the individual has been identified as in need of treatment for an HBV-related condition. In certain embodiments provided herein are methods for prophylactically reducing HBV expression in an individual. Certain embodiments include treating an individual in need thereof by administering to an individual a therapeutically effective amount of an antisense compound targeted to an HBV nucleic acid.

Due to overlapping transmission routes, many people have been exposed to both hepatitis B virus (HBV) and hepatitis C virus (HCV), and a smaller proportion are chronically infected with both viruses, especially in regions such as Asia where HBV is endemic. Estimates suggest that up to 10% of people with HCV may also have HBV, while perhaps 20% of people with HBV are co-infected with HCV. However, treatment of hepatitis B or hepatitis B in HBV-HCV co-infected individuals has not been well studied. Treatment is complicated by the fact that HCV and HBV appear to inhibit each other's replication (though not all studied have observed this interaction). Therefore, treatment that fully suppresses HBV could potentially allow HCV to re-emerge, or vice versa. Therefore, the compounds and compositions described herein may advantageously be used for treating patients infected with both HBV and HCV. Exemplary treatment options for hepatitis C(HCV) include interferons, e.g., interferon alpha-2b, interferon alpha-2a, and interferon alphacon-1. Less frequent interferon dosing can be achieved using pegylated interferon (interferon attached to a polyethylene glycol moiety which significantly improves its pharmacokinetic profile). Combination therapy with interferon alpha-2b (pegylated and unpegylated) and ribavirin has also been shown to be efficacious for some patient populations. Other agents currently being developed include HCV RNA replication inhibitors (e.g., ViroPharma's VP50406 series), HCV antisense agents, HCV therapeutic vaccines, HCV protease inhibitors, HCV helicase inhibitors and HCV antibody therapy (monoclonal or polyclonal).

In certain embodiments, treatment with the methods, compounds, and compositions described herein is useful for preventing an HBV-related condition associated with the presence of the hepatitis B virus. In certain embodiments, treatment with the methods, compounds, and compositions described herein is useful for preventing an HBV-related condition.

In one embodiment, administration of a therapeutically effective amount of an antisense compound targeted to an HBV nucleic acid is accompanied by monitoring of HBV levels in the serum of an individual to determine an individual's response to administration of the antisense compound. In certain embodiments, administration of a therapeutically effective amount of an antisense compound targeted to an HBV nucleic acid is accompanied by monitoring of HBV RNA levels in the serum of an individual to determine an individual's response to administration of the antisense compound. In certain embodiments, administration of a therapeutically effective amount of an antisense compound targeted to an HBV nucleic acid is accompanied by monitoring of HBV DNA levels in the serum of an individual to determine an individual's response to administration of the antisense compound. In certain embodiments, administration of a therapeutically effective amount of an antisense compound targeted to an HBV nucleic acid is accompanied by monitoring of HBV S antigen (HBsAg) levels in the serum of an individual to determine an individual's response to administration of the antisense compound. In certain embodiments, administration of a therapeutically effective amount of an antisense compound targeted to an HBV nucleic acid is accompanied by monitoring of HBV E antigen (HBeAg) levels in the serum of an individual to determine an individual's response to administration of the antisense compound. An individual's response to administration of the antisense compound is used by a physician to determine the amount and duration of therapeutic intervention.

In certain embodiments, administration of an antisense compound targeted to an HBV nucleic acid results in reduction of HBV expression by at least 15, 20, 25, 30, 35, 40, 45, 50, 55, 60, 65, 70, 75, 80, 85, 90, 95 or 99%, or a range defined by any two of these values. In certain embodiments, administration of an antisense compound targeted to an HBV nucleic acid results in reduced symptoms associated with the HBV-related condition and reduced HBV-related markers in the blood. In certain embodiments, administration of an HBV antisense compound decreases HBV RNA levels, HBV DNA levels, HBsAg levels, or HBeAg levels by at least 15, 20, 25, 30, 35, 40, 45, 50, 55, 60, 65, 70, 75, 80, 85, 90, 95 or 99%, or a range defined by any two of these values.

In certain embodiments, pharmaceutical compositions comprising an antisense compound targeted to HBV are used for the preparation of a medicament for treating a patient suffering or susceptible to an HBV-related condition.

### Certain Combination Therapies

In certain embodiments, one or more pharmaceutical compositions provided herein are co-administered with one or more other pharmaceutical agents. In certain embodiments, such one or more other pharmaceutical agents are designed to treat the same disease, disorder, or condition as the one or more pharmaceutical compositions provided herein. In certain embodiments, such one or more other pharmaceutical agents are designed to treat a different disease, disorder, or condition as the one or more pharmaceutical compositions provided herein. In certain embodiments, such one or more other pharmaceutical agents are designed to treat an undesired side effect of one or more pharmaceutical compositions provided herein. In certain embodiments, one or more pharmaceutical compositions provided herein are co-administered with another pharmaceutical agent to treat an undesired effect of that other pharmaceutical agent. In certain embodiments, one or more pharmaceutical compositions provided herein are co-administered with another pharmaceutical agent to produce a combinational effect. In certain embodiments, one or more pharmaceutical compositions provided herein are co-administered with another pharmaceutical agent to produce a synergistic effect.

In certain embodiments, one or more pharmaceutical compositions provided herein and one or more other pharmaceutical agents are administered at the same time. In certain embodiments, one or more pharmaceutical compositions provided herein and one or more other pharmaceutical agents are administered at different times. In certain embodiments, one or more pharmaceutical compositions provided herein and one or more other pharmaceutical agents are prepared together in a single formulation. In certain embodiments, one or more pharmaceutical compositions provided herein and one or more other pharmaceutical agents are prepared separately. In certain embodiments the antisense oligonucleotides disclosed is administered in combination with an HCV agent. In further embodiments, the HCV compound is administered simultaneously as the antisense compound; in other embodiments, the HCV compound is administered separately; so that a dose of each of the HCV agent and the antisense compound overlap, in time, within the patient's body. In related embodiments, the HCV agent may be selected from interferon alpha-2b, interferon alpha-2a, and interferon alphacon-1 (pegylated and unpegylated); ribavirin; an HCV RNA replication inhibitor (e.g., ViroPharma's VP50406 series); an HCV antisense agent; an HCV therapeutic vaccine; an HCV protease inhibitor; an HCV helicase inhibitor; and an HCV antibody therapy (monoclonal or polyclonal).

In other embodiments, an HBV antisense compound of the present invention may be administered to a patient infected with HBV, in combination with one or more HBV therapeutic agents, wherein the one or more HBV therapeutic agents may be administered in the same drug formulation as the HBV ASO compound, or may be administered in a separate formulation. The one or more HBV therapeutic agents may be administered simultaneously with the ASO HBV compound, or may be administered separately, so that a dose of each of the HBV ASO compound and the HBV therapeutic agent overlap, in time, within the patient's body. In related embodiments, the one or more HBV therapeutic agent may be selected from interferon alpha-2b, interferon alpha-2a, and interferon alphacon-1 (pegylated and unpegylated), ribavirin; an HBV RNA replication inhibitor; a second HBV antisense compound; an HBV therapeutic vaccine; an HBV prophylactic vaccine; lamivudine (3TC); entecavir; tenofovir; telbivudine (LdT); adefovir; and an HBV antibody therapy (monoclonal or polyclonal).

### EXAMPLES

### Non-Limiting Disclosure and Incorporation by Reference

While certain compounds, compositions and methods described herein have been described with specificity in accordance with certain embodiments, the following examples serve only to illustrate the compounds described herein and are not intended to limit the same. Each of the references recited in the present application is incorporated herein by reference in its entirety.

### Example 1

### Dose-Dependent Inhibition of Viral HBV RNA by Antisense Oligonucleotides Targeted to the Hepatitis B Virus Gene Sequence

Antisense chimeric antisense oligonucleotides were designed to target the Hepatitis B virus gene sequence. The chimeric antisense oligonucleotides in Table 1 were designed as 2-10-2 LNA or 3-10-3 LNA gapmers. One antisense oligonucleotide, ISIS 510167, was designed with deoxyribose and LNA units interspersed throughout its length, as displayed in the table. The 2-10-2 gapmers are 14 nucleosides in length, wherein the central gap segment is comprised of ten 2′-deoxyribose nucleosides and is flanked on both sides (in the 5′ and 3′ directions) by wings comprising two locked nucleic acids (LNA) each. The 3-10-3 gapmers are 16 nucleosides in length, wherein the central gap segment is comprised of ten 2′-deoxyribose nucleosides and is flanked on both sides (in the 5′ and 3′ directions) by wings comprising three locked nucleic acids (LNA) each. The internucleoside linkages throughout each gapmer are phosphorothioate (P═S) linkages. All cytosine residues throughout each gapmer are 5-methylcytosines. Each antisense oligonucleotide in Table 1 is targeted to SEQ ID NO: 1 (GENBANK Accession No. U95551.1). ‘Start site’ indicates the 5′-most nucleoside to which the gapmer is targeted in the viral gene sequence. ‘Stop site’ indicates the 3′-most nucleoside to which the gapmer is targeted in the viral gene sequence. The subscripts represent the following chemistry motifs: ‘d’=2′-deoxyribose; ‘l’=LNA; ‘s’=thioate ester. The symbol ‘m’=2′-O-methylribose.

A 4-8-4 gapmer, ISIS 510166, was included in the assay as a comparator. The newly designed gapmers were tested at various doses in HepG2.2.15 cells. Cells were plated at a density of 25,000 cells per well and transfected using electroporation with 740 nM, 2,222 nM, 6,666 nM, and 20,0000 nM concentrations of antisense oligonucleotide, as specified in Table 2. After a treatment period of approximately 16 hours, RNA was isolated from the cells and HBV mRNA levels were measured by quantitative real-time PCR. Viral primer probe set RTS3370 (forward sequence CTTGGTCATGGGCCATCAG, designated herein as SEQ ID NO: 15; reverse sequence CGGCTAGGAGTTCCGCAGTA, designated herein as SEQ ID NO: 16; probe sequence TGCGTGGAACCTTTTCGGCTCC, designated herein as SEQ ID NO: 17) was used to measure mRNA levels. RTS3370 detects the full length mRNA and the second portions of the pre-S1, pre-S2 and pre-C mRNA transcripts. HBV mRNA levels were adjusted according to total RNA content, as measured by RIBOGREEN®. Results are presented as percent inhibition of HBV, relative to untreated control cells.

The half maximal inhibitory concentration (IC₅₀) of each oligonucleotide is also presented in Table 2 and was calculated by plotting the concentrations of oligonucleotides used versus the percent inhibition of HBV mRNA expression achieved at each concentration, and noting the concentration of oligonucleotide at which 50% inhibition of HBV mRNA expression was achieved compared to the control. As illustrated in Table 2, HBV mRNA levels were significantly reduced in a dose-dependent manner in antisense oligonucleotide treated cells.

TABLE 1
LNA gapmers targeted to SEQ ID NO: 1

| | | | | | SEQ |
|---|---|---|---|---|---|
| | Start | Stop | | | ID |
| ISIS No | Site | Site | Sequence (5′ to 3′) | Motif | NO |
| 510148 | 258 | 273 | GₗₛAₗₛGₗₛA_{ds}G_{ds}A_{ds}A_{ds}G_{ds}T_{ds}mC_{ds}mC_{ds}A_{ds}mC_{ds}mCₗₛAₗₛmCₗ | 3-10-3 | 18 |
| 510149 | 259 | 274 | TₗₛGₗₛAₗₛGdsA_{ds}G_{ds}A_{ds}A_{ds}G_{ds}T_{ds}mC_{ds}mC_{ds}A_{ds}mCₗₛmCₗₛAₗ | 3-10-3 | 19 |
| 510150 | 414 | 429 | GₗₛAₗₛGₗₛG_{ds}mC_{ds}A_{ds}T_{ds}A_{ds}G_{ds}mC_{ds}A_{ds}G_{ds}mC_{ds}AₗₛGₗₛGₗ | 3-10-3 | 20 |
| 510151 | 415 | 430 | TₗₛGₗₛAₗₛG_{ds}G_{ds}mC_{ds}A_{ds}T_{ds}A_{ds}G_{ds}mC_{ds}A_{ds}G_{ds}mCₗₛAₗₛGₗ | 3-10-3 | 21 |
| 510152 | 417 | 432 | GₗₛAₗₛTₗₛG_{ds}A_{ds}G_{ds}G_{ds}mC_{ds}A_{ds}T_{ds}A_{ds}G_{ds}mC_{ds}AₗₛGₗₛmCₗ | 3-10-3 | 22 |
| 510153 | 602 | 617 | GₗₛAₗₛTₗₛG_{ds}G_{ds}G_{ds}A_{ds}T_{ds}G_{ds}G_{ds}G_{ds}A_{ds}A_{ds}TₗₛAₗₛmCₗ | 3-10-3 | 23 |
| 510154 | 639 | 654 | GₗₛGₗₛmCₗₛmC_{ds}mC_{ds}A_{ds}mC_{ds}T_{ds}mC_{ds}mC_{ds}mC_{ds}A_{ds}T_{ds}AₗₛGₗₛGₗ | 3-10-3 | 24 |
| 510155 | 640 | 655 | AₗₛGₗₛGₗₛmC_{ds}mC_{ds}mC_{ds}A_{ds}mC_{ds}T_{ds}mC_{ds}mC_{ds}mC_{ds}A_{ds}TₗₛAₗₛGₗ | 3-10-3 | 25 |
| 510156 | 643 | 658 | mCₗₛTₗₛGₗₛA_{ds}G_{ds}G_{ds}mC_{ds}mC_{ds}mC_{ds}A_{ds}mC_{ds}T_{ds}mC_{ds}mCₗₛmCₗₛAₗ | 3-10-3 | 26 |
| 510157 | 259 | 272 | AₗₛGₗₛA_{ds}G_{ds}A_{ds}A_{ds}G_{ds}T_{ds}mC_{ds}mC_{ds}A_{ds}mC_{ds}mCₗₛAₗ | 2-10-2 | 27 |
| 510158 | 260 | 273 | GₗₛAₗₛG_{ds}A_{ds}G_{ds}A_{ds}A_{ds}G_{ds}T_{ds}mC_{ds}mC_{ds}A_{ds}mCₗₛmCₗ | 2-10-2 | 28 |
| 510159 | 415 | 428 | AₗₛGₗₛG_{ds}mC_{ds}A_{ds}T_{ds}A_{ds}G_{ds}mC_{ds}A_{ds}G_{ds}mC_{ds}AₗₛGₗ | 2-10-2 | 29 |
| 510160 | 416 | 429 | GₗₛAₗₛGdsGdsmCdsAdsTdsAdsGdsmCdsAdsGdsmCₗₛAₗ | 2-10-2 | 30 |
| 510161 | 418 | 431 | AₗₛTₗₛG_{ds}A_{ds}G_{ds}G_{ds}mC_{ds}A_{ds}T_{ds}A_{ds}G_{ds}C_{ds}AₗₛGₗ | 2-10-2 | 31 |
| 510162 | 603 | 616 | AₗₛTₗₛG_{ds}G_{ds}G_{ds}A_{ds}T_{ds}G_{ds}G_{ds}G_{ds}A_{ds}A_{ds}TₗₛAₗ | 2-10-2 | 32 |
| 510163 | 640 | 653 | GₗₛmCₗₛmC_{ds}mC_{ds}A_{ds}mC_{ds}T_{ds}mC_{ds}mC_{ds}mC_{ds}A_{ds}T_{ds}AₗₛGₗ | 2-10-2 | 33 |
| 510164 | 641 | 654 | GₗₛGₗₛmC_{ds}mC_{ds}mC_{ds}A_{ds}mC_{ds}T_{ds}mC_{ds}mC_{ds}mC_{ds}A_{ds}TₗₛAₗ | 2-10-2 | 34 |
| 510165 | 644 | 657 | TₗₛGₗₛA_{ds}G_{ds}G_{ds}mC_{ds}mC_{ds}mC_{ds}A_{ds}mC_{ds}T_{ds}mC_{ds}mCₗₛmCₗ | 2-10-2 | 35 |
| 510166 | 414 | 429 | GₗₛAₗₛGₗₛGₗₛmC_{ds}A_{ds}T_{ds}A_{ds}G_{ds}mC_{ds}A_{ds}G_{ds}mCₗₛAₗₛGₗₛGₗ | 4-8-4 | 20 |
| 510167 | 640 | 655 | AₗₛGₗₛGₗₛmCₗₛmC_{ds}mC_{ds}A_{ds}mC_{ds}T_{ds}mC_{ds}mC_{ds}mCₗₛA_{ds}TₗₛAₗₛGₗ | 4-7-11-3 | 25 |

TABLE 2
Dose-dependent antisense inhibition of HBV RNA in
HepG2.2.15cells using electroporation

| | | | | | IC₅₀ |
|---|---|---|---|---|---|
| ISIS No | 740 nM | 2222 nM | 6666 nM | 20000 nM | (μM) |
| 510148 | 34 | 40 | 48 | 77 | 3.9 |
| 510149 | 34 | 43 | 57 | 64 | 4.1 |
| 510150 | 39 | 53 | 71 | 79 | 1.7 |
| 510151 | 30 | 53 | 79 | 85 | 1.9 |
| 510152 | 40 | 47 | 67 | 82 | 2.0 |
| 510153 | 39 | 53 | 70 | 76 | 1.7 |
| 510154 | 29 | 35 | 45 | 53 | 14.1 |
| 510155 | 21 | 39 | 48 | 62 | 7.0 |
| 510156 | 33 | 40 | 56 | 60 | 5.3 |
| 510157 | 19 | 26 | 46 | 57 | 10.8 |
| 510158 | 13 | 35 | 44 | 55 | 11.5 |
| 510159 | 29 | 45 | 58 | 72 | 3.5 |
| 510160 | 20 | 41 | 64 | 66 | 4.5 |
| 510161 | 13 | 33 | 49 | 70 | 6.3 |
| 510162 | 26 | 35 | 49 | 61 | 7.4 |
| 510163 | 29 | 28 | 39 | 43 | >20.0 |
| 510164 | 24 | 23 | 29 | 29 | >20.0 |
| 510165 | 28 | 31 | 46 | 58 | 10.5 |
| 510166 | 34 | 38 | 42 | 57 | 11.1 |
| 510167 | 15 | 30 | 25 | 18 | >20.0 |

### Example 2

### Dose-Dependent Inhibition of Viral HBV RNA in HepAD38 Cells

The gapmers from the study described in Example 1 were tested at various doses in human hepatoma HepAD38 cells, in which HBV production is under the control of a tetracycline-regulated promoter. Cells were plated at a density of 45,000 cells per well and transfected using electroporation with 740 nM, 2,222 nM, 6,666 nM, and 20,0000 nM concentrations of antisense oligonucleotide, as specified in Table 3. After a treatment period of approximately 16 hours, RNA was isolated from the cells and HBV mRNA levels were measured by quantitative real-time PCR. Viral primer probe set RTS3372 (forward sequence ATCCTATCAACACTTCCGGAAACT, designated herein as SEQ ID NO: 36; reverse sequence CGACGCGGCGATTGAG, designated herein as SEQ ID NO: 37; probe sequence AAGAACTCCCTCGCCTCGCAGACG, designated herein as SEQ ID NO: 38) was used to measure mRNA levels. RTS3372 detects the full length genomic sequence. HBV mRNA levels were adjusted according to total RNA content, as measured by RIBOGREEN®. Results are presented as percent inhibition of HBV, relative to untreated control cells.

The half maximal inhibitory concentration (IC₅₀) of each oligonucleotide is also presented in Table 3. As illustrated in Table 3, HBV mRNA levels were significantly reduced in a dose-dependent manner in antisense oligonucleotide treated cells.

TABLE 3
Dose-dependent antisense inhibition of HBV RNA in
HepAD38 cells using electroporation and RTS3372

| | | | | | IC₅₀ |
|---|---|---|---|---|---|
| ISIS No | 740 nM | 2222 nM | 6666 nM | 20000 nM | (μM) |
| 510148 | 22 | 10 | 75 | 65 | 5.7 |
| 510149 | 29 | 30 | 54 | 61 | 7 |
| 510150 | 33 | 74 | 60 | 18 | 1.9 |
| 510151 | 0 | 54 | 4 | 10 | >20.0 |
| 510152 | 39 | 65 | 70 | 79 | 1.2 |
| 510153 | 37 | 50 | 79 | 89 | 1.7 |
| 510154 | 7 | 25 | 59 | 75 | 5.7 |
| 510155 | 9 | 40 | 48 | 65 | 6.9 |
| 510156 | 34 | 69 | 59 | 60 | 1.6 |
| 510157 | 0 | 0 | 14 | 28 | >20.0 |
| 510158 | 6 | 34 | 45 | 67 | 7.6 |
| 510159 | 10 | 58 | 74 | 83 | 2.8 |
| 510160 | 16 | 59 | 69 | 38 | 7.7 |
| 510161 | 6 | 43 | 57 | 82 | 4.4 |
| 510162 | 3 | 34 | 54 | 71 | 6.1 |
| 510163 | 0 | 0 | 17 | 46 | >20.0 |
| 510164 | 0 | 0 | 54 | 55 | 11.7 |
| 510165 | 0 | 4 | 41 | 55 | 14.6 |
| 510166 | 0 | 30 | 54 | 72 | 6.7 |
| 510167 | 0 | 13 | 20 | 33 | >20.0 |

The mRNA levels were also measured using the RTS3373MGB primer probe set (forward sequence CCGACCTTGAGGCATACTTCA, designated herein as SEQ ID NO: 39; reverse sequence AATTTATGCCTACAGCCTCCTAGTACA, designated herein as SEQ ID NO: 40; probe sequence TTAAAGACTGGGAGGAGTTG, designated herein as SEQ ID NO: 41). RTS3373MGB detects the full length mRNA and the second portions of the pre-S1, pre-S2, pre-C, and pre-X mRNA transcripts. The results are presented in Table 4.

TABLE 4
Dose-dependent antisense inhibition of HBV RNA in
HepAD38 cells using electroporation and RTS3373MGB

| ISIS No | 740 nM | 2222 nM | 6666 nM | 20000 nM | IC₅₀ (μM) |
|---|---|---|---|---|---|
| 510148 | 0 | 0 | 54 | 61 | 13.1 |
| 510149 | 0 | 26 | 56 | 56 | 9.0 |
| 510150 | 0 | 20 | 52 | 63 | 8.8 |
| 510151 | 0 | 30 | 43 | 62 | 9.5 |
| 510152 | 19 | 38 | 53 | 79 | 4.5 |
| 510153 | 32 | 38 | 68 | 83 | 2.8 |
| 510154 | 11 | 34 | 30 | 53 | 19.8 |
| 510155 | 4 | 14 | 18 | 59 | 21.8 |
| 510156 | 18 | 34 | 49 | 60 | 8.1 |
| 510157 | 6 | 14 | 24 | 42 | >20.0 |
| 510158 | 31 | 34 | 37 | 64 | 9.5 |
| 510159 | 0 | 41 | 66 | 92 | 4.0 |
| 510160 | 0 | 37 | 51 | 71 | 6.6 |
| 510161 | 0 | 25 | 30 | 68 | 10.2 |
| 510162 | 32 | 20 | 52 | 63 | 7.8 |
| 510163 | 0 | 0 | 6 | 14 | >20.0 |
| 510164 | 0 | 0 | 5 | 21 | >20.0 |
| 510165 | 0 | 0 | 29 | 40 | >20.0 |
| 510166 | 0 | 7 | 9 | 50 | >20.0 |
| 510167 | 0 | 1 | 5 | 0 | >20.0 |

1. A compound comprising a modified oligonucleotide consisting of 10 to 30 linked nucleosides and having a nucleobase sequence comprising at least 8 contiguous nucleobases of any of the nucleobase sequences of SEQ ID NOs: 18-35, wherein said modified oligonucleotide is chimeric and wherein said modified oligonucleotide is at least 95% complementary to SEQ ID NO: 1.

2. The compound of claim 1, wherein said modified oligonucleotide is at least 99% complementary to SEQ ID NO: 1.

3. The compound of claim 1, wherein said modified oligonucleotide is 100% complementary to SEQ ID NO: 1.

4. The compound of claim 1, consisting of a single-stranded modified oligonucleotide.

5. The compound of claim 1, wherein at least one internucleoside linkage is a modified internucleoside linkage.

6. The compound of claim 5, wherein each internucleoside linkage is a phosphorothioate internucleoside linkage.

7. The compound of claim 1, wherein at least one nucleoside of the modified oligonucleotide comprises a modified sugar.

8. The compound of claim 7, wherein the at least one modified sugar is a bicyclic sugar.

9. (canceled)

10. The compound of claim 8, wherein the bicyclic sugar comprises a 4′-CH₂—O-2′ group.

11. The compound of claim 1, wherein at least one nucleoside comprises a modified nucleobase.

12. The compound of claim 11, wherein the modified nucleobase is a 5-methylcytosine.

13. The compound of claim 1, wherein the modified oligonucleotide comprises:
a gap segment consisting of linked deoxynucleosides;
a 5′ wing segment consisting of linked nucleosides; and
a 3′ wing segment consisting of linked nucleosides;
wherein the gap segment is positioned between the 5′ wing segment and the 3′ wing segment and wherein each nucleoside of each wing segment comprises a modified sugar.

14. The compound of claim 13, wherein the modified oligonucleotide comprises:
a gap segment consisting of ten linked deoxynucleosides;
a 5′ wing segment consisting of three linked nucleosides; and
a 3′ wing segment consisting of three linked nucleosides;
wherein the gap segment is positioned between the 5′ wing segment and the 3′ wing segment and wherein each nucleoside of each wing segment comprises a modified sugar.

15. The compound of claim 13, wherein the modified oligonucleotide comprises:
a gap segment consisting of ten linked deoxynucleosides;
a 5′ wing segment consisting of two linked nucleosides; and
a 3′ wing segment consisting of two linked nucleosides;
wherein the gap segment is positioned between the 5′ wing segment and the 3′ wing segment and wherein each nucleoside of each wing segment comprises a modified sugar.

16. The compound of claim 13, wherein the modified oligonucleotide comprises:
a gap segment consisting of ten linked deoxynucleosides;
a 5′ wing segment consisting of two linked nucleosides; and
a 3′ wing segment consisting of two linked nucleosides;
wherein the gap segment is positioned between the 5′ wing segment and the 3′ wing segment and wherein each nucleoside of each wing segment comprises a bicyclic sugar.

17. The compound of claim 13, wherein the modified oligonucleotide comprises:
a gap segment consisting of ten linked deoxynucleosides;
a 5′ wing segment consisting of three linked nucleosides; and
a 3′ wing segment consisting of three linked nucleosides;
wherein the gap segment is positioned between the 5′ wing segment and the 3′ wing segment, wherein each nucleoside of each wing segment comprises a 4′-CH₂—O-2′ bridge; and wherein each internucleoside linkage is a phosphorothioate linkage.

18. The compound of claim 1, wherein the modified oligonucleotide consists of 16 linked nucleosides.

19. The compound of claim 1, wherein the modified oligonucleotide consists of 14 linked nucleosides.

20. The compound of claim 1, wherein said modified oligonucleotide consists of 15 to 30 linked nucleosides.

21. A compound comprising a modified oligonucleotide consisting of 10 to 30 linked nucleosides comprising an at least 8 consecutive nucleobase portion complementary to nucleotides 258-274, 258-273, 259-272, 260-273, 414-429, 414-432, 414-430, 415-430, 417-432, 415-432, 415-428, 416-429, 416-426, 418-431, 602-617, 603-616, 639-654, 639-658, 639-655, 640-658, 640-653, 640-655, 641-654, 643-658, or 644-657 of SEQ ID NO: 1, wherein said modified oligonucleotide is at least 90% complementary to SEQ ID NO: 1.

22. A composition comprising the compound of claim 1 or salt thereof and at least one of a pharmaceutically acceptable carrier or diluent.

23. (canceled)

24. (canceled)

25. A method of preventing, treating, ameliorating, or slowing the progression of a HBV-related disease, disorder or condition in an animal comprising administering to the animal the compound of claim 1, wherein administering the compound prevents, treats, ameliorates, or slows progression of a HBV-related disease, disorder or condition.

26. The method of claim 25, wherein the disease, disorder or condition is liver disease.

27. The method of claim 26, wherein the disease, disorder or condition is jaundice, liver inflammation, liver fibrosis, inflammation, liver cirrhosis, liver failure, diffuse hepatocellular inflammatory disease, hemophagocytic syndrome, serum hepatitis, HBV viremia, or liver disease-related transplantation.

28. The method of claim 26, wherein the disease or condition is a hyperproliferative condition.

29. The method of claim 28, wherein the hyperproliferative condition is liver cancer.

30. A method of reducing antigen levels in an animal comprising administering to said animal the compound of claim 1, wherein HBsAg levels or HBeAg levels are reduced.

31-38. (canceled)


SEQUENCE LISTING (41 sequences)

SEQ ID NO: 1 - DNA, 3182 bp, Hepatitis B virus
aattccacaacctttcaccaaactctgcaagatcccagagtgagaggcctgtatttccctgctggtggctccagttcaggagcagtaaaccctgttccgactactgcctctcccttatcgtcaatcttctcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcctgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaaccaccagcacgggaccatgccgaacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctgaattttatgggttatgtcattggaagttatgggtccttgccacaagaacacatcatacaaaaaatcaaagaatgttttagaaaacttcctattaacaggcctattgattggaaagtatgtcaacgaattgtgggtcttttgggttttgctgccccatttacacaatgtggttatcctgcgttaatgcccttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcgtgcgtggaaccttttcggctcctctgccgatccatactgcggaactcctagccgcttgttttgctcgcagcaggtctggagcaaacattatcgggactgataactctgttgtcctctcccgcaaatatacatcgtatccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccgaatgttgcccaaggtcttacataagaggactcttggactctctgcaatgtcaacgaccgaccttgaggcatacttcaaagactgtttgtttaaagactgggaggagttgggggaggagattagattaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcatctagagacctagtagtcagttatgtcaacactaatatgggcctaaagttcaggcaactcttgtggtttcacatttcttgtctcacttttggaagagaaaccgttatagagtatttggtgtctttcggagtgtggattcgcactcctccagcttatagaccaccaaatgcccctatcctatcaacacttccggaaactactgttgttagacgacgaggcaggtcccctagaagaagaactccctcgcctcgcagacgaaggtctcaatcgccgcgtcgcagaagatctcaatctcgggaacctcaatgttagtattccttggactcataaggtggggaactttactggtctttattcttctactgtacctgtctttaatcctcattggaaaacaccatcttttcctaatatacatttacaccaagacattatcaaaaaatgtgaacagtttgtaggcccacttacagttaatgagaaaagaagattgcaattgattatgcctgctaggttttatccaaaggttaccaaatatttaccattggataagggtattaaaccttattatccagaacatctagttaatcattacttccaaactagacactatttacacactctatggaaggcgggtatattatataagagagaaacaacacatagcgcctcattttgtgggtcaccatattcttgggaacaagatctacagcatggggcagaatctttccaccagcaatcctctgggattctttcccgaccaccagttggatccagccttcagagcaaacacagcaaatccagattgggacttcaatcccaacaaggacacctggccagacgccaacaaggtaggagctggagcattcgggctgggtttcaccccaccgcacggaggccttttggggtggagccctcaggctcagggcatactacaaactttgccagcaaatccgcctcctgcctccaccaatcgccagacaggaaggcagcctaccccgctgtctccacctttgagaaacactcatcctcaggccatgcagtgg

SEQ ID NO: 2 - DNA, 20 bp, Artificial Sequence
  Synthetic Oligonucleotide
cctgctggtggctccagttc

SEQ ID NO: 3 - DNA, 42 bp, Artificial Sequence
  Synthetic Oligonucleotide
agagtctagactcgtggtggacttctctcaattttctagggg

SEQ ID NO: 4 - DNA, 26 bp, Artificial Sequence
  Synthetic Oligonucleotide
tggatgtgtctgcggcgttttatcat

SEQ ID NO: 5 - DNA, 27 bp, Artificial Sequence
  Synthetic Oligonucleotide
catcctgctgctatgcctcatcttctt

SEQ ID NO: 6 - DNA, 17 bp, Artificial Sequence
  Synthetic Oligonucleotide
caaggtatgttgcccgt

SEQ ID NO: 7 - DNA, 17 bp, Artificial Sequence
  Synthetic Oligonucleotide
tgtattcccatcccatc

SEQ ID NO: 8 - DNA, 20 bp, Artificial Sequence
  Synthetic Oligonucleotide
cctatgggagtgggcctcag

SEQ ID NO: 9 - DNA, 19 bp, Artificial Sequence
  Synthetic Oligonucleotide
tggctcagtttactagtgc

SEQ ID NO: 10 - DNA, 17 bp, Artificial Sequence
  Synthetic Oligonucleotide
gggctttcccccactgt

SEQ ID NO: 11 - DNA, 30 bp, Artificial Sequence
  Synthetic Oligonucleotide
tcctctgccgatccatactgcggaactcct

SEQ ID NO: 12 - DNA, 19 bp, Artificial Sequence
  Synthetic Oligonucleotide
cgcacctctctttacgcgg

SEQ ID NO: 13 - DNA, 17 bp, Artificial Sequence
  Synthetic Oligonucleotide
ggagtgtggattcgcac

SEQ ID NO: 14 - DNA, 20 bp, Artificial Sequence
  Synthetic Oligonucleotide
gaagaagaactccctcgcct

SEQ ID NO: 15 - DNA, 19 bp, Artificial Sequence
  Primer
cttggtcatgggccatcag

SEQ ID NO: 16 - DNA, 20 bp, Artificial Sequence
  Primer
cggctaggagttccgcagta

SEQ ID NO: 17 - DNA, 22 bp, Artificial Sequence
  Probe
tgcgtggaaccttttcggctcc

SEQ ID NO: 18 - DNA, 16 bp, Artificial Sequence
  Synthetic Oligonucleotide
gagagaagtccaccac

SEQ ID NO: 19 - DNA, 16 bp, Artificial Sequence
  Synthetic Oligonucleotide
tgagagaagtccacca

SEQ ID NO: 20 - DNA, 16 bp, Artificial Sequence
  Synthetic Oligonucleotide
gaggcatagcagcagg

SEQ ID NO: 21 - DNA, 16 bp, Artificial Sequence
  Synthetic Oligonucleotide
tgaggcatagcagcag

SEQ ID NO: 22 - DNA, 16 bp, Artificial Sequence
  Synthetic Oligonucleotide
gatgaggcatagcagc

SEQ ID NO: 23 - DNA, 16 bp, Artificial Sequence
  Synthetic Oligonucleotide
gatgggatgggaatac

SEQ ID NO: 24 - DNA, 16 bp, Artificial Sequence
  Synthetic Oligonucleotide
ggcccactcccatagg

SEQ ID NO: 25 - DNA, 16 bp, Artificial Sequence
  Synthetic Oligonucleotide
aggcccactcccatag

SEQ ID NO: 26 - DNA, 16 bp, Artificial Sequence
  Synthetic Oligonucleotide
ctgaggcccactccca

SEQ ID NO: 27 - DNA, 14 bp, Artificial Sequence
  Synthetic Oligonucleotide
agagaagtccacca

SEQ ID NO: 28 - DNA, 14 bp, Artificial Sequence
  Synthetic Oligonucleotide
gagagaagtccacc

SEQ ID NO: 29 - DNA, 14 bp, Artificial Sequence
  Synthetic Oligonucleotide
aggcatagcagcag

SEQ ID NO: 30 - DNA, 14 bp, Artificial Sequence
  Synthetic Oligonucleotide
gaggcatagcagca

SEQ ID NO: 31 - DNA, 14 bp, Artificial Sequence
  Synthetic Oligonucleotide
atgaggcatagcag

SEQ ID NO: 32 - DNA, 14 bp, Artificial Sequence
  Synthetic Oligonucleotide
atgggatgggaata

SEQ ID NO: 33 - DNA, 14 bp, Artificial Sequence
  Synthetic Oligonucleotide
gcccactcccatag

SEQ ID NO: 34 - DNA, 14 bp, Artificial Sequence
  Synthetic Oligonucleotide
ggcccactcccata

SEQ ID NO: 35 - DNA, 14 bp, Artificial Sequence
  Synthetic Oligonucleotide
tgaggcccactccc

SEQ ID NO: 36 - DNA, 24 bp, Artificial Sequence
  Primer
atcctatcaacacttccggaaact

SEQ ID NO: 37 - DNA, 16 bp, Artificial Sequence
  Primer
cgacgcggcgattgag

SEQ ID NO: 38 - DNA, 24 bp, Artificial Sequence
  Probe
aagaactccctcgcctcgcagacg

SEQ ID NO: 39 - DNA, 21 bp, Artificial Sequence
  Primer
ccgaccttgaggcatacttca

SEQ ID NO: 40 - DNA, 27 bp, Artificial Sequence
  Primer
aatttatgcctacagcctcctagtaca

SEQ ID NO: 41 - DNA, 20 bp, Artificial Sequence
  Probe
ttaaagactgggaggagttg